(19) United States
(12) Patent Application Publication
(10) Pub. No.: US 2019/0161480 A1
(43) Pub. Date: May 30, 2019

(54) Chemical Compounds

(71) Applicant: GLAXOSMITHKLINE INTELLECTUAL PROPERTY DEVELOPMENT LIMITED, Brentford, Middlesex (GB)

(72) Inventors: Sebastien Andre CAMPOS, Stevenage, Hertfordshire (GB); Samuel Edward DALTON, Stevenage, Hertfordshire (GB); Vipulkumar Kantibhai PATEL, Stevenage, Hertfordshire (GB)

(73) Assignee: GLAXOSMITHKLINE INTELLECTUAL PROPERTY DEVELOPMENT LIMITED, Brentford, Middlesex (GB)

(21) Appl. No.: 16/323,529

(22) PCT Filed: Aug. 7, 2017
(86) PCT No.: PCT/EP2017/069886

§ 371 Date: Feb. 6, 2019

(30) Foreign Application Priority Data
Aug. 8, 2016 (GB) 1613557.6

(51) Int. Cl. C07D 413/14; A61P 11/06

(52) U.S. Cl. CPC C07D 413/14; A61P 11/06

(57) ABSTRACT

The invention is directed to certain novel compounds. Specifically, the invention is directed to compounds of formula (I):

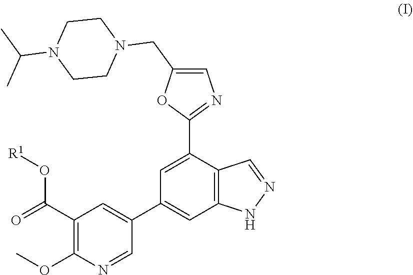

and salts thereof. The compounds of the invention are inhibitors of kinase activity, in particular PI3-kinase activity.

### FIELD OF THE INVENTION

The present invention is directed to compounds which are inhibitors of kinase activity, pharmaceutical compositions comprising the compounds, and the use of the compounds or the compositions in the treatment of various disorders. More specifically, the compounds of the invention are inhibitors of the activity or function of the phosphoinositide 3′OH kinase family (hereinafter PI3-kinases), for example PI3Kδ, PI3Kα, PI3Kβ and/or PI3Kγ.

### BACKGROUND OF THE INVENTION

Cellular membranes represent a large store of second messengers that can be enlisted in a variety of signal transduction pathways. In relation to function and regulation of effector enzymes in phospholipids signalling pathways, class I PI3-kinases (e.g. PI3Kdelta) generate second messengers from the membrane phospholipid pools. Class I PI3Ks convert the membrane phospholipid PI(4,5)P₂ into PI(3,4,5)P₃, which functions as a second messenger. PI and PI(4)P are also substrates of PI3K and can be phosphorylated and converted into PI3P and PI(3,4)P₂, respectively. In addition, these phosphoinositides can be converted into other phosphoinositides by 5′-specific and 3′-specific phosphatases. Thus, PI3K enzymatic activity results either directly or indirectly in the generation of two 3′-phosphoinositide subtypes which function as second messengers in intracellular signal transduction pathways (Trends Biochem. Sci. 22(7) p. 267-72 (1997) by Vanhaesebroeck et al.; Chem. Rev. 101(8) p. 2365-80 (2001) by Leslie et al.; Annu. Rev. Cell Dev. Biol. 17 p. 615-75 (2001) by Katso et al.; and Cell. Mol. Life Sci. 59(5) p. 761-79 (2002) by Toker). To date, eight mammalian PI3Ks have been identified, divided into three main classes (I, II, and III) on the basis of sequence homology, structure, binding partners, mode of activation, and substrate preference. In vitro, class I PI3Ks can phosphorylate phosphatidylinositol (PI), phosphatidylinositol-4-phosphate (PI4P), and phosphatidylinositol-4,5-bisphosphate (PI(4,5)P₂) to produce phosphatidylinositol-3-phosphate (PI3P), phosphatidylinositol-3,4-bisphosphate (PI(3,4)P₂, and phosphatidylinositol-3,4,5-trisphosphate (PI(3,4,5)P₃, respectively. Class II PI3Ks can phosphorylate PI and PI4P. Class III PI3Ks can only phosphorylate PI (Vanhaesebroeck et al. (1997), above; Vanhaesebroeck et al. Exp. Cell Res. 253(1) p. 239-54 (1999); and Leslie et al. (2001), above).

Class I PI3K is a heterodimer consisting of a p110 catalytic subunit and a regulatory subunit, and the family is further divided into class Ia and class Ib enzymes on the basis of regulatory partners and mechanism of regulation. Class Ia enzymes consist of three distinct catalytic subunits (p110α, p110β, and p110δ) that dimerise with five distinct regulatory subunits (p85α, p55α, p50α, p85β, and p55γ), with all catalytic subunits being able to interact with all regulatory subunits to form a variety of heterodimers. Class Ia PI3K are generally activated in response to growth factor-stimulation of receptor tyrosine kinases, via interaction of the regulatory subunit SH2 domains with specific phospho-tyrosine residues of the activated receptor or adaptor proteins such as IRS-1. Small GTPases (ras as an example) are also involved in the activation of PI3K in conjunction with receptor tyrosine kinase activation. Both p110α and p110β are constitutively expressed in all cell types, whereas p110δ expression is more restricted to leukocyte populations and some epithelial cells. In contrast, the single Class Ib enzyme consists of a p110γ catalytic subunit that interacts with a p101 regulatory subunit. Furthermore, the Class Ib enzyme is activated in response to G-protein coupled receptor (GPCR) systems and its expression appears to be limited to leukocytes.

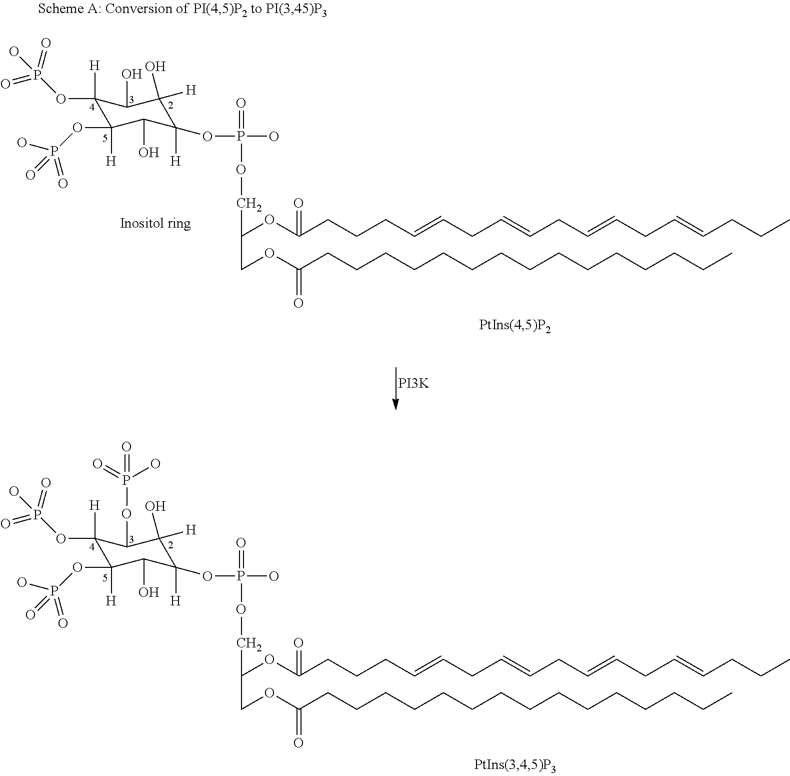

As illustrated in Scheme A above, phosphoinositide 3-kinases (PI3Ks) phosphorylate the hydroxyl of the third carbon of the inositol ring. The phosphorylation of phosphoinositides to generate PtdIns(3,4,5)P₃, PtdIns(3,4)P₂ and PtdIns(3)P, produces second messengers for a variety of signal transduction pathways, including those essential to cell proliferation, cell differentiation, cell growth, cell size, cell survival, apoptosis, adhesion, cell motility, cell migration, chemotaxis, invasion, cytoskeletal rearrangement, cell shape changes, vesicle trafficking and metabolic pathway (Katso et al. (2001), above; and Mol. Med. Today 6(9) p. 347-57 (2000) by Stein et al.).

The activity of PI3-kinases responsible for generating these phosphorylated signalling products was originally identified as being associated with viral oncoproteins and growth factor receptor tyrosine kinases that phosphorylate phosphatidylinositol (PI) and its phosphorylated derivatives at the 3′-hydroxyl of the inositol ring (Panayotou et al. Trends Cell Biol. 2 p. 358-60 (1992)). However, more recent biochemical studies have revealed that class I PI3-kinases (e.g. class IA isoform PI3Kδ) are dual-specific kinase enzymes, meaning they display both lipid kinase (phosphorylation of phosphoinositides) as well as protein kinase activity, which have been shown to be capable of phosphorylation of other protein as substrates, including auto-phosphorylation as an intramolecular regulatory mechanism (EMBO J. 18(5) p. 1292-302 (1999) by Vanhaesebroeck et al.). Cellular processes in which PI3Ks play an essential role include suppression of apoptosis, reorganization of the actin skeleton, cardiac myocyte growth, glycogen synthase stimulation by insulin, TNFα-mediated neutrophil priming and superoxide generation, and leukocyte migration and adhesion to endothelial cells.

PI3-kinase activation, is believed to be involved in a wide range of cellular responses including cell growth, differentiation, and apoptosis (Parker, Current Biology 5(6) p. 577-79 (1995); and Yao et al. Science 267(5206) p. 2003-06 (1995)). PI3-kinase appears to be involved in a number of aspects of leukocyte activation. A p85-associated PI3-kinase has been shown to physically associate with the cytoplasmic domain of CD28, which is an important costimulatory molecule for the activation of T-cells in response to antigen (Pagès et al. Nature 369 p. 327-29 (1994); and Rudd, Immunity 4 p. 527-34 (1996)). Activation of T cells through CD28 lowers the threshold for activation by antigen and increases the magnitude and duration of the proliferative response. These effects are linked to increases in the transcription of a number of genes including interleukin-2 (IL2), an important T cell growth factor (Fraser et al. Science 251(4991) p. 313-16 (1991)).

PI3Kγ has been identified as a mediator of G beta-gamma-dependent regulation of JNK activity, and G beta-gamma are subunits of heterotrimeric G proteins (Lopez-Ilasaca et al. J. Biol. Chem. 273(5) p. 2505-8 (1998)). Recently, (Laffargue et al. Immunity 16(3) p. 441-51 (2002)) it has been described that PI3Kγ relays inflammatory signals through various G(i)-coupled receptors and is central to mast cell function, stimuli in the context of leukocytes, and immunology including cytokines, chemokines, adenosines, antibodies, integrins, aggregation factors, growth factors, viruses or hormones for example (J. Cell Sci. 114 (Pt 16) p. 2903-10 (2001) by Lawlor et al.; Laffargue et al. (2002), above; and Curr. Opinion Cell Biol. 14(2) p. 203-13 (2002) by Stephens et al.).

Specific inhibitors against individual members of a family of enzymes provide invaluable tools for deciphering functions of each enzyme. Two compounds, LY294002 and wortmannin (hereinafter), have been widely used as PI3-kinase inhibitors. These compounds are non-specific PI3K inhibitors, as they do not distinguish among the four members of Class I PI3-kinases. For example, the IC₅₀ values of wortmannin against each of the various Class I PI3-kinases are in the range of 1-10 nM. Similarly, the IC₅₀ values for LY294002 against each of these PI3-kinases is about 15-20 μM (Fruman et al. Ann. Rev. Biochem. 67 p. 481-507 (1998)), also 5-10 microM on CK2 protein kinase and some inhibitory activity on phospholipases. Wortmannin is a fungal metabolite which irreversibly inhibits PI3K activity by binding covalently to the catalytic domain of this enzyme. Inhibition of PI3K activity by wortmannin eliminates subsequent cellular response to the extracellular factor. For example, neutrophils respond to the chemokine fMet-Leu-Phe (fMLP) by stimulating PI3K and synthesizing PtdIns (3, 4, 5)P₃. This synthesis correlates with activation of the respiratory burst involved in neutrophil destruction of invading microorganisms. Treatment of neutrophils with wortmannin prevents the fMLP-induced respiratory burst response (Thelen et al. Proc. Natl. Acad. Sci. USA 91 p. 4960-64 (1994)). Indeed, these experiments with wortmannin, as well as other experimental evidence, show that PI3K activity in cells of hematopoietic lineage, particularly neutrophils, monocytes, and other types of leukocytes, is involved in many of the non-memory immune response associated with acute and chronic inflammation.

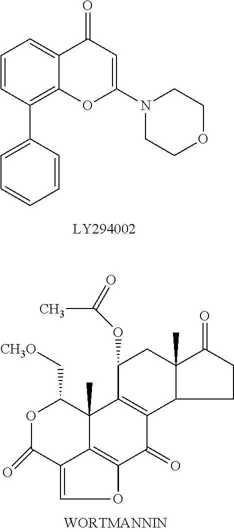

Based on studies using wortmannin, there is evidence that PI3-kinase function is also required for some aspects of leukocyte signaling through G-protein coupled receptors (Thelen et al. (1994), above). Moreover, it has been shown that wortmannin and LY294002 block neutrophil migration and superoxide release.

It is now well understood that deregulation of oncogenes and tumour suppressor genes contributes to the formation of malignant tumours, for example by way of increased cell growth and proliferation or increased cell survival. It is also now known that signalling pathways mediated by the PI3K family have a central role in a number of cell processes including proliferation and survival, and deregulation of these pathways is a causative factor a wide spectrum of human cancers and other diseases (Katso et al. Annual Rev. Cell Dev. Biol. (2001) 17 p. 615-675 and Foster et al. J. Cell Science (2003) 116(15) p. 3037-3040). PI3K effector proteins initiate signalling pathways and networks by translocating to the plasma membrane through a conserved Pleckstrin Homology (PH) domain, which specifically interacts with PtdIns(3,4,5)P3 (Vanhaesebroeck et al. Annu. Rev. Biochem. (2001) 70 p. 535-602). The effector proteins signalling through PtdIns(3,4,5)P3 and PH domains include Serine/Threonine (Ser/Thr) kinases, Tyrosine kinases, Rac or Arf GEFs (Guanine nucleotide exchange factors) and Arf GAPs (GTPase activating proteins).

In B and T cells PI3Ks have an important role through activation of the Tec family of protein tyrosine kinases which include Bruton's tyrosine kinase (BTK) in B cells and Interleukin-2-inducible T-cell kinase (ITK) in T cells. Upon PI3K activation, BTK or ITK translocate to the plasma membrane where they are subsequently phosphorylated by Src kinases. One of the major targets of activated ITK is phospholipase C-gamma (PLCγ1), which hydrolyses PtdIns(4,5)P2 into Ins(3,4,5)P3 and initiates an intracellular increase in calcium levels and diacylglycerol (DAG) which can activate Protein Kinases C in activated T cells.

Unlike the Class IA p110α and p110β, p110δ is expressed in a tissue restricted fashion. Its high expression level in lymphocytes and lymphoid tissues suggests a role in PI3K-mediated signalling in the immune system. The p110δ kinase dead knock-in mice are also viable and their phenotype is restricted to defects in immune signalling (Okkenhaug et al. Science (2002) 297 p. 1031-4). These transgenic mice have offered insight into the function of PI3Kδ in B-cell and T-cell signalling. In particular, p110δ is required for PtdIns(3,4,5)P3 formation downstream of CD28 and/or T cell Receptor (TCR) signalling. A key effect of PI3K signalling downstream of TCR is the activation of Akt, which phosphorylates anti-apoptotic factors as well as various transcription factors for cytokine production. As a consequence, T cells with inactive p110δ have defects in proliferation and Th1 and Th2 cytokine secretion. Activation of T cells through CD28 lowers the threshold for TCR activation by antigen and increases the magnitude and duration of the proliferative response. These effects are mediated by the PI3Kδ-dependent increase in the transcription of a number of genes including IL2, an important T cell growth factor.

Therefore, PI3K inhibitors are anticipated to provide therapeutic benefit via its role in modulating T-cell mediated inflammatory responses associated to respiratory diseases such as asthma, COPD and cystic fibrosis. In addition, there is indication that T-cell directed therapies may provide corticosteroid sparing properties (Alexander et al. Lancet (1992) 339 p. 324-8) suggesting that it may provide a useful therapy either as a standalone or in combination with inhaled or oral glucocorticosteroids in respiratory diseases. A PI3K inhibitor might also be used alongside other conventional therapies such as a long acting beta-agonist (LABA) or leukotriene antagonist in asthma.

In the vasculature, PI3Kδ is expressed by endothelial cells and participates in neutrophil trafficking by modulating the proadhesive state of these cells in response to TNFalpha (Puri et al. Blood (2004) 103(9) p. 3448-56.). A role for PI3Kδ in TNFalpha-induced signalling of endothelial cells is demonstrated by the pharmacological inhibition of Akt phosphorylation and PDK1 activity. In addition, PI3Kδ is implicated in vascular permeability and airway tissue edema through the VEGF pathway (Lee et al. J. Allergy Clin. Immunol. (2006) 118(2) p. 403-9). These observations suggest additional benefits of PI3Kδ inhibition in asthma by the combined reduction of leukocyte extravasation and vascular permeability associated with asthma. In addition, PI3Kδ activity is required for mast cell function both in vitro and in vivo (Ali et al. Nature (2004) 431 p. 1007-11; and Ali et al. J. Immunol. (2008) 180(4) p. 2538-44) further suggesting that PI3K inhibition should be of therapeutical benefit for allergic indications such asthma, allergic rhinitis and atopic dermatitis.

The role of PI3Kδ in B cell proliferation, antibody secretion, B-cell antigen and IL-4 receptor signalling, B-cell antigen presenting function is also well established Okkenhaug et al. (2002), above; Al-Alwan et al. J. Immunol. (2007) 178(4) p. 2328-35; and Bilancio et al. Blood (2006) 107(2) p. 642-50) and indicates a role in autoimmune diseases such as rheumatoid arthritis or systemic lupus erythematosus (SLE). Therefore PI3K inhibitors may also be of benefit for these indications.

Pharmacological inhibition of PI3Kδ inhibits fMLP-dependent neutrophil chemotaxis on an ICAM coated agarose matrix integrin-dependent biased system (Sadhu et al., J. Immunol. (2003) 170(5) p. 2647-54.). Inhibition of PI3Kδ regulates neutrophil activation, adhesion and migration without affecting neutrophil mediated phagocytosis and bactericidal activity over Staphylococcus aureus (Sadhu et al. Biochem. Biophys. Res. Commun. (2003) 308(4) p. 764-9). Overall, the data suggest that PI3Kδ inhibition should not globally inhibit neutrophil functions required for innate immune defence. PI3Kδ's role in neutrophils offers further scope for treating inflammatory diseases involving tissue remodeling such as COPD or rheumatoid arthritis.

PI3Kδ inhibition may also lead to cancer immunotherapy. For instance, PI3Kδ has a critical signalling role in regulatory T cells (Tregs), which enables their expansion (Patton et al. PLoS One. 2011; 6(3):e17359). Activation of Tregs is one of the key processes that allow cancer cells to build immunological tolerance and escape immune surveillance. Another aspect of cancer immunity where PI3Kδ inhibitors may play a role is in upregulating the expression of PD-L1 (Programmed cell death 1 ligand 1) as has been shown in cultured airway epithelial cells (Kan-0 et al. Biochem Biophys Res Commun. 2013; 435(2):195-201). PD-L1, expressed on various cell types such as T and B lymphocytes, NK and DC cells or epithelial cells, is involved in suppressing T cell dependent immunity such as the activation of cytotoxic CD8 T cells. Neutralising antibodies targeting PD-L1 are currently being developed as cancer immuno-therapeutics. Therefore, PI3Kδ inhibition may provide a novel way of enhancing anti-tumour responses. A similar rationale may also be applied to anti-infective immunity where the balance of Tregs and CD8s are known to play an important role in the outcome of the immune response such as viral infections.

The central nervous system (CNS) is also enriched with PI3Kδ expression (Eickholt et al. PLoS One 2007 11; 2(9):e869). A more recent report further uncovered a link between PI3Kδ and the neuregulin NRC-1 and ErbB4 receptor in the CNS with implications for schizophrenia (Law et al. Proc Natl Acad Sci USA. 2012; 109(30):12165-70). It was previously known that increased expression of a splice variant of ErbB4 containing the cytoplasmic portion, Cyt1, resulted in activation of the PI3K pathway as well as increased risk of schizophrenia. The publication by Law et al. indicates that the schizophrenia genetically associated Cyt1 couples preferentially to the PI3Kδ isoform. Furthermore, the PI3Kδ selective inhibitor, IC87114, showed remarkable efficacy in a mouse model of amphetamine-induced psychosis (Law et al. Proc Natl Acad Sci USA. 2012; 109(30):12165-70). Therefore PI3Kδ inhibitors have the potential to form the basis for new schizophrenia therapy approaches.

In addition, there is also good evidence that class IA PI3K enzymes also contribute to tumourigenesis in a wide variety of human cancers, either directly or indirectly (Vivanco and Sawyers, Nature Reviews Cancer (2002) 2(7) p. 489-501). For example, inhibition of PI3Kδ may have a therapeutic role for the treatment of malignant haematological disorders such as acute myeloid leukaemia (Billottet et al. Oncogene (2006) 25(50) p. 6648-59). Moreover, activating mutations within p110α (PIK3CA gene) have been associated with various other tumours such as those of the colon and of the breast and lung (Samuels et al. Science (2004) 304(5670) p. 554).

It has also been shown that PI3K is involved in the establishment of central sensitization in painful inflammatory conditions (Pezet et al. The J. of Neuroscience (2008) 28 (16) p. 4261-4270).

A wide variety of retroviruses and DNA based viruses activate the PI3K pathway as a way of preventing host cell death during viral infection and ultimately exploiting the host cell synthesis machinery for its replication (Virology 344(1) p. 131-8 (2006) by Vogt et al.; and Nat. Rev. Microbiol. 6(4) p. 265-75 (2008) by Buchkovich et al.). Therefore PI3K inhibitors may have anti-viral properties in addition to more established oncolytic and anti-inflammatory indications. These antiviral effects raise interesting prospects in viral induced inflammatory exacerbations. For example, the common cold human rhinovirus (HRV) is responsible for more than 50% of respiratory tract infections but complications of these infections can be significant in certain populations. This is particularly the case in respiratory diseases such as asthma or chronic obstruction pulmonary disease (COPD). Rhinoviral infection of epithelial cells leads to a PI3K dependent cytokine and chemokine secretion (J. Biol. Chem. (2005) 280(44) p. 36952 by Newcomb et al.). This inflammatory response correlates with worsening of respiratory symptoms during infection. Therefore PI3K inhibitors may dampen an exaggerated immune response to an otherwise benign virus. The majority of HRV strains infect bronchial epithelial cells by initially binding to the ICAM-1 receptor. The HRV-ICAM-1 complex is then further internalised by endocytosis and it has been shown that this event requires PI3K activity (J. Immunol. (2008) 180(2) p. 870-880 by Lau et al.). Therefore, PI3K inhibitors may also block viral infections by inhibiting viral entry into host cells.

PI3K inhibitors may be useful in reducing other types of respiratory infections including the fungal infection aspergillosis (Mucosal Immunol. (2010) 3(2) p. 193-205 by Bonifazi et al.). In addition, PI3Kδ deficient mice are more resistant towards infections by the protozoan parasite Leishmania major (J. Immunol. (2009) 183(3) p. 1921-1933 by Liu et al.) or by the intracellular bacteria Listeria (Pearce et al. J. Immunol. (2015) 195(7) p. 3206-17). Taken with effects on viral infections, these reports suggest that PI3K inhibitors may be useful for the treatment of a wide variety of infections.

A published report points towards PI3Kδ inhibitors having potential benefits in preventing infections by the common airway bacterial pathogen S. Pneumoniae (Fallah et al., Mech. Ageing Dev. 2011; 132(6-7): 274-86). In this report PI3Kδ is shown to reduce the macrophage-derived cytokines required to mount an effective antibody response to S. pneumoniae in the elderly. The anti-bacterial benefit of PI3Kδ inhibitors may thus be useful in the treatment of bacterial respiratory tract infections and bacterial exacerbations of respiratory conditions and lung damage such as asthma, COPD and cystic fibrosis, and pneumonia.

PI3K inhibition has also been shown to promote regulatory T cell differentiation (Proc. Natl. Acad. Sci. USA (2008) 105(22) p. 7797-7802 by Sauer et al.) suggesting that PI3K inhibitors may serve therapeutic purposes in auto-immune or allergic indications by inducing immuno-tolerance towards self antigen or allergen. The PI3Kδ isoform has also been linked to smoke induced glucocorticoid insensitivity (Am. J. Respir. Crit. Care Med. (2009) 179(7) p. 542-548 by Marwick et al.). This observation suggests that COPD patients, which otherwise respond poorly to corticosteroids, may benefit from the combination of a PI3K inhibitor with a corticosteroid.

PI3K has also been involved in other respiratory conditions such as idiopathic pulmonary fibrosis (IPF). IPF is a fibrotic disease with progressive decline of lung function and increased mortality due to respiratory failure. In IPF, circulating fibrocytes are directed to the lung via the chemokine receptor CXCR4. PI3K is required for both signalling and expression of CXCR4 (Int. J. Biochem. and Cell Biol. (2009) 41 p. 1708-1718 by Mehrad et al.). Therefore, by reducing CXCR4 expression and blocking its effector function, a PI3K inhibitor should inhibit the recruitment of fibrocytes to the lung and consequently slow down the fibrotic process underlying IPF, a disease with high unmet need.

A number of selective reversible PI3Kδ inhibitors have been developed, most notably Zydelig™, which has recently been approved by the FDA for the treatment of chronic lymphocytic leukemia. Wortmannin and its structurally related analogues (e.g. PX-866) are, to date, the only reported PI3Kδ inhibitors which covalently bind to the kinase via reaction with a conserved lysine residue situated in the ATP binding site. However, these compounds show poor selectivity, especially for the various PI3K isoforms, restricting their use to specific indications.

The present inventors believe that by modifying selective reversible PI3Kδ inhibitors with carefully positioned electrophilic moieties, selective irreversible PI3Kδ inhibition may be achieved, despite the conserved nature of the targeted lysine residue within the PI3K family.

Compounds which are PI3-kinase inhibitors may be useful in the treatment of disorders associated with inappropriate kinase activity, in particular inappropriate PI3-kinase activity, for example in the treatment and prevention of disorders mediated by PI3-kinase mechanisms. Such disorders include respiratory diseases including asthma, chronic obstructive pulmonary disease (COPD) and idiopathic pulmonary fibrosis (IPF); ciliopathy including primary ciliary dyskinesia, polycystic liver disease and nephronophthisis; bacterial infections including bacterial respiratory tract infections, for example infections by S. Pneumoniae, H. Influenzae, M. Catarrhalis and/or mycobacteria such as Mycobacterium tuberculosis, and bacterial exacerbations of respiratory conditions and lung damage such as asthma, COPD and cystic fibrosis; viral infections including viral respiratory tract infections, for example infections by influenza, rhinovirus, respiratory syncytial virus (RSV), human parainfluenza virus (HPIV), adenovirus and/or coronavirus, and viral exacerbation of respiratory conditions and lung damage such as asthma, COPD and cystic fibrosis; other non-viral respiratory infections including aspergillosis and leishmaniasis; allergic diseases including allergic rhinitis, atopic dermatitis and psoriasis; autoimmune diseases including ankylosing spondylitis, Churg-Strauss syndrome, Crohn's disease, Glomerulonephritis, Henoch-Schonlein purpura, idiopathic thrombocytopenic purpura (ITP), interstitial cystitis, pemphigus, primary sclerosing cholangitis, psoriasis, rheumatoid arthritis, sarcoidosis, Sjogren's syndrome, Type 1 diabetes, ulcerative colitis, vasculitis and Wegener's granulomatosis; inflammatory disorders including inflammatory bowel disease; diabetes; cardiovascular diseases including thrombosis, atherosclerosis and hypertension; hematologic malignancies; neurodegenerative diseases; pancreatitis; multiorgan failure; kidney diseases; platelet aggregation; cancer; sperm motility; transplantation rejection; graft rejection; lung injuries; pain including pain associated with rheumatoid arthritis or osteoarthritis, back pain, general inflammatory pain, post hepatic neuralgia, diabetic neuropathy, inflammatory neuropathic pain (trauma), trigeminal neuralgia and Central pain; fibrotic diseases; depression; and psychotic disorders including schizophrenia.

In one embodiment, compounds of the invention may show selectivity for PI3-kinases over other kinases.

In another embodiment, compounds of the invention may be potent inhibitors of PI3Kδ.

In another embodiment, compounds of the invention may show selectivity for PI3Kδ over other PI3-kinases.

In a further embodiment, compounds of the invention may be selective irreversible inhibitors of PI3Kδ.

### SUMMARY OF THE INVENTION

The invention is directed to certain novel compounds. Specifically, the invention is directed to compounds of formula (I)

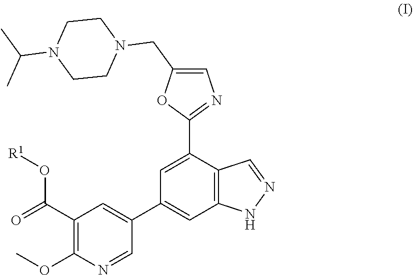

wherein R¹ is as defined below, and salts thereof.

The compounds are inhibitors of kinase activity, in particular PI3-kinase activity. Compounds which are PI3-kinase inhibitors may be useful in the treatment of disorders associated with inappropriate PI3-kinase activity. Accordingly, the invention is further directed to pharmaceutical compositions comprising a compound of formula (I) or a pharmaceutically acceptable salt thereof, and one or more pharmaceutically acceptable excipients. The invention is still further directed to methods of treating disorders mediated by inappropriate PI3-kinase activity comprising administering a safe and effective amount of a compound of formula (I) or a pharmaceutically acceptable salt thereof, to a patient in need thereof.

### BRIEF DESCRIPTION OF THE FIGURES

FIG. 1 shows protein mass spectrometry for Example 7 with PI3Kδ. Full modification was observed within 5 min in a 2:1 inhibitor:protein ratio, and no additional adducts were observed after 20 h with a 10:1 inhibitor:protein ratio. Preincubation of the enzyme with a potent ATP-competitive inhibitor attentuated formation of this adduct. Control compound (reversibly binding acid Example 2) showed no modification of the protein.

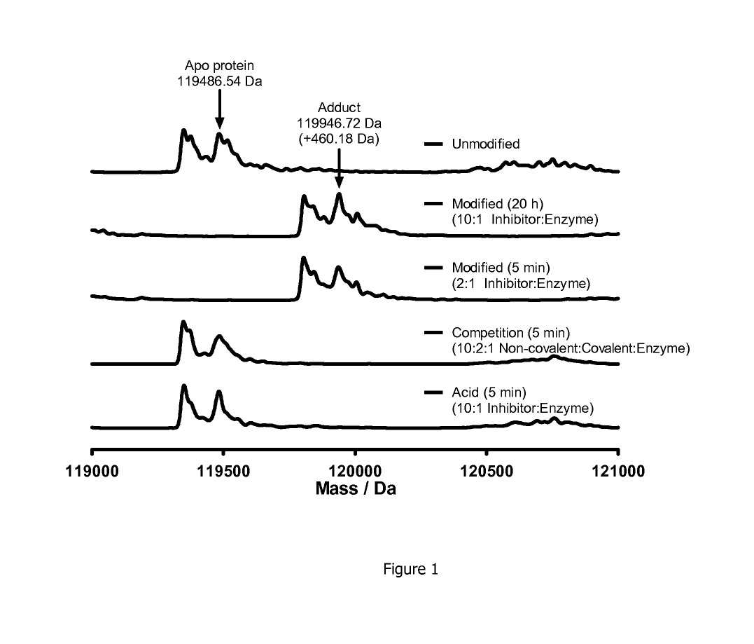

FIG. 2 shows a jump dilution assay with PI3Kδ. Recovery of enzyme activity was observed for the control compounds Example 1 (closed diamonds) and Example 2 (open diamonds). No recovery of activity, consistent with irreversible inhibition was observed for Example 7 (open squares), in good accordance with known irreversible inactivator, Wortmannin (crosses) relative to no inhibitor control (open circles).

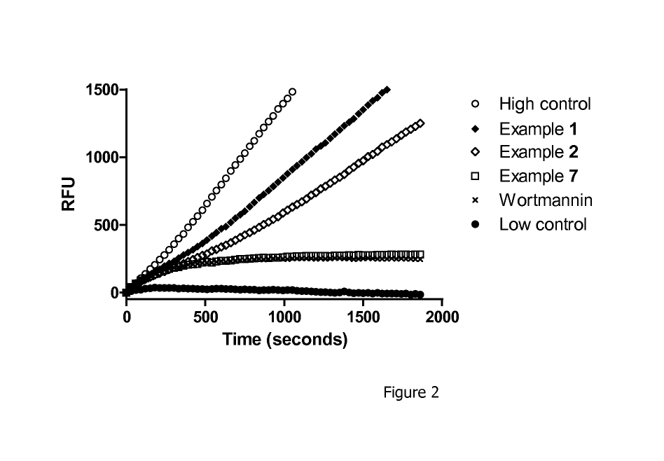

FIG. 3 shows the cellular wash out data for Example 7. The IC₅₀ curve 48 h after washing cells to remove compound followed, closely the curve without washing the cells. This confirms a duration of action of at least 48 h for Example 7 at PI3Kδ in CD4+ T cells, supporting an irreversible covalent mode of action.

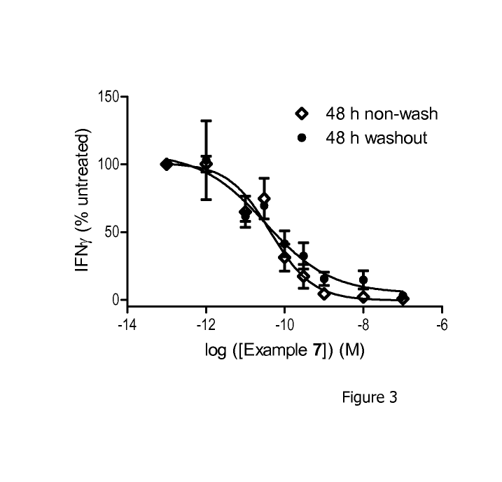

### DETAILED DESCRIPTION OF THE INVENTION

In one embodiment, the invention is directed to compounds of formula (I)

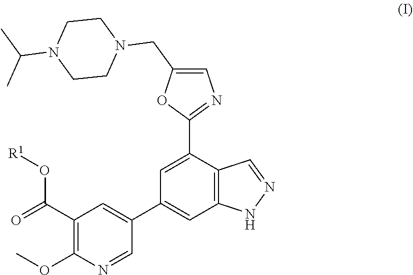

wherein

R¹ is hydrogen, C₁₋₆alkyl or phenyl, wherein the C₁₋₆alkyl is optionally substituted by —CF₃ and the phenyl is optionally substituted by one or two substituents independently selected from C₁₋₆alkyl, C₁₋₆alkoxy, halogen, —CN, —CF₃, —CO₂R², —CO₂NHR³, —NR⁴R⁵, —NO₂ and —SF₅;

R² to R⁵ are each independently selected from hydrogen and C₁₋₆alkyl;

and salts thereof (hereinafter “compounds of the invention”).

In one embodiment, R¹ is hydrogen, methyl or phenyl, wherein the methyl is optionally substituted by —CF₃ and the phenyl is optionally substituted by one or two substituents independently selected from C₁₋₆alkyl, C₁₋₆alkoxy, halogen, —CN, —CF₃, —CO₂R², —CO₂NHR³, —NR⁴R⁵, —NO₂ and —SF₅. In another embodiment, R¹ is hydrogen, methyl or phenyl, wherein the methyl is optionally substituted by —CF₃ and the phenyl is optionally substituted by one or two substituents independently selected from C₁₋₆alkyl, C₁₋₆alkoxy, halogen, —CF₃ and —NO₂. In another embodiment, R¹ is methyl optionally substituted by —CF₃. In another embodiment, R¹ is phenyl optionally substituted by one or two substituents independently selected from C₁₋₆alkyl, C₁₋₆alkoxy, halogen, —CN, —CF₃, —CO₂R², —CO₂NHR³, —NR⁴R⁵, —NO₂ and —SF₅. In another embodiment, R¹ is phenyl optionally substituted by one or two substituents independently selected from C₁₋₆alkyl, C₁₋₆alkoxy, halogen, —CF₃ and —NO₂. In another embodiment, R¹ is phenyl. In another embodiment, R¹ is phenyl substituted by two substituents independently selected from C₁₋₆alkyl. In another embodiment, R¹ is phenyl substituted by C₁₋₆alkoxy. In another embodiment, R¹ is phenyl substituted by halogen, for example fluoro. In another embodiment, R¹ is phenyl substituted by —NO₂. In another embodiment, R¹ is phenyl substituted by —CF₃.

In another embodiment, R¹ is phenyl optionally substituted by one or two substituents wherein the substituents are in the ortho and/or para position.

It is to be understood that the present invention covers all combinations of substituent groups described hereinabove.

Compounds of the invention include the compounds of Examples 1 to 11 and salts thereof.

In one embodiment, the compound of the invention is:
methyl 5-(4-(5-((4-isopropylpiperazin-1-yl)methyl)oxazol-2-yl)-1H-indazol-6-yl)-2-methoxynicotinate;
5-(4-(5-((4-isopropylpiperazin-1-yl)methyl)oxazol-2-yl)-1H-indazol-6-yl)-2-methoxynicotinic acid;
2-nitrophenyl 5-(4-(5-((4-isopropylpiperazin-1-yl)methyl)oxazol-2-yl)-1H-indazol-6-yl)-2-methoxynicotinate;
4-nitrophenyl 5-(4-(5-((4-isopropylpiperazin-1-yl)methyl)oxazol-2-yl)-1H-indazol-6-yl)-2-methoxynicotinate;
2-(trifluoromethyl)phenyl 5-(4-(5-((4-isopropylpiperazin-1-yl)methyl) oxazol-2-yl)-1H-indazol-6-yl)-2-methoxynicotinate;
4-(trifluoromethyl)phenyl 5-(4-(5-((4-isopropylpiperazin-1-yl)methyl)oxazol-2-yl)-1H-indazol-6-yl)-2-methoxynicotinate;
4-fluorophenyl 5-(4-(5-((4-isopropylpiperazin-1-yl)methyl)oxazol-2-yl)-1H-indazol-6-yl)-2-methoxynicotinate;
phenyl 5-(4-(5-((4-isopropylpiperazin-1-yl)methyl)oxazol-2-yl)-1H-indazol-6-yl)-2-methoxynicotinate;
2,4-dimethylphenyl 5-(4-(5-((4-isopropylpiperazin-1-yl)methyl)oxazol-2-yl)-1H-indazol-6-yl)-2-methoxynicotinate;
4-methoxyphenyl 5-(4-(5-((4-isopropylpiperazin-1-yl)methyl)oxazol-2-yl)-1H-indazol-6-yl)-2-methoxynicotinate;
2,2,2-trifluoroethyl 5-(4-(5-((4-isopropylpiperazin-1-yl)methyl)oxazol-2-yl)-1H-indazol-6-yl)-2-methoxynicotinate;
   or a salt thereof.

### Terms and Definitions

“Alkyl” refers to a saturated hydrocarbon chain having the specified number of member atoms. For example, C₁₋₆alkyl refers to an alkyl group having from 1 to 6 member atoms, for example from 1 to 4 member atoms. Alkyl groups may be straight or branched. Representative branched alkyl groups have one, two, or three branches. Alkyl groups may be optionally substituted with one or more substituents as defined herein. Alkyl includes methyl, ethyl, propyl (n-propyl and isopropyl), butyl (n-butyl, isobutyl, and t-butyl), pentyl (n-pentyl, isopentyl, and neopentyl), and hexyl. Alkyl groups may also be part of other groups, for example C₁₋₆alkoxy.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” refers to products whose enantiomeric excess is 99% ee or greater.

“Half-life” (or “half-lives”) refers to the time required for half of a quantity of a substance to be converted to another chemically distinct species in vitro or in vivo.

“Halogen” refers to the halogen radical fluoro, choro, bromo or iodo. In one embodiment, halogen is fluoro.

“Optionally substituted” indicates that a group may be unsubstituted or substituted with one or more substituents as defined herein.

“Substituted” in reference to a group indicates that a hydrogen atom attached to a member atom within a group is replaced. It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. Suitable substituents are defined herein for each substituted or optionally substituted group.

“Pharmaceutically acceptable” refers to those compounds, salts, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
2-MeTHF 2-Methyltetrahydrofuran
Ac Acetyl
COD 1,5-Cyclooctadiene
CPME Cyclopentyl methyl ether
DAD Diode array detector
DCM Dichloromethane
DHP 3,4-Dihydro-2H-pyran
DIPEA N, N-Diisopropylethylamine
DMF N, N-Dimethylformamide
DMSO Dimethyl sulfoxide
DTT Dithiothreitol
Et Ethyl
EtOAc Ethyl acetate
EtOH Ethanol
g Grams
h Hour(s)
i.d. Internal diameter
IPA Isopropanol
iPr Isopropyl
HATU 1-[Bis(dimethylamino)methylene]-1H-1,2,3-triazolo[4,5-b]pyridinium 3-oxid hexafluorophosphate
HPLC High performance liquid chromatography
Kg Kilograms
L Litre
LCMS Liquid chromatography mass spectroscopy
μL Microlitres
μM Micromolar
μmol Micromoles
M Molar
MDAP Mass-directed automated preparative HPLC
Me Methyl
MeCN Acetonitrile
MeOH Methanol
mg Milligrams
MIBK Methyl isobutyl ketone
min Minute(s)
mL Millilitres
mol Moles
mM Millimolar
mmol Millimoles
nM Nanomolar
OAc Acetate
nM Nanomolar
nm Nanometres
NMR Nuclear magnetic resonance
PdCl₂(dppf) [1,1′-Bis(diphenylphosphino)ferrocene]dichloropalladium(II)
Pin Pinacol
pM Picomolar
PyBOP (Benzotriazol-1-yloxy)tripyrrolidinophosphonium hexafluorophosphate
Rₜ Retention time
TFA Trifluoroacetic acid
THF Tetrahydrofuran
TMS Trimethylsilane
UV Ultraviolet
XPhos 2-dicyclohexylphosphino-2′,4′,6′-triisopropylbiphenyl
XRPD X-ray powder diffraction

Included within the scope of the “compounds of the invention” are all polymorphs, radiolabelled derivatives, stereoisomers and optical isomers of the compounds of formula (I) and salts thereof.

The compounds of the invention may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically acceptable solvates may be formed wherein solvent molecules are incorporated into the crystalline lattice during crystallization. The compounds of the invention may exist in solvated and unsolvated form. Solvates may involve nonaqueous solvents such as ethanol, isopropanol, DMSO, acetic acid, ethanolamine, and EtOAc, or they may involve water as the solvent that is incorporated into the crystalline lattice. Solvates wherein water is the solvent that is incorporated into the crystalline lattice are typically referred to as “hydrates”. Hydrates include stoichiometric hydrates as well as compositions containing variable amounts of water.

The skilled artisan will further appreciate that certain compounds of the invention that exist in crystalline form, including the various solvates thereof, may exhibit polymorphism (i.e. the capacity to occur in different crystalline structures). These different crystalline forms are typically known as “polymorphs”. The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The invention also includes isotopically-labelled compounds, which are identical to the compounds of the invention, but for the fact that one or more atoms are replaced by an atom having an atomic mass or mass number different from the atomic mass or mass number most commonly found in nature. Examples of isotopes that can be incorporated into the compounds of the invention include isotopes of hydrogen, carbon, nitrogen, oxygen and fluorine, such as ²H, ³H, ¹¹C, ¹⁴C and ¹⁸F.

The compounds of the invention may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in a compound of the invention, or in any chemical structure illustrated herein, is not specified the structure is intended to encompass any stereoisomer and all mixtures thereof. Thus, compounds of the invention containing one or more chiral center may be used as racemic mixtures, enantiomerically enriched mixtures, or as enantiomerically pure individual stereoisomers.

Individual stereoisomers of a compound of the invention which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The compounds of the invention may also contain centers of geometric asymmetry. Where the stereochemistry of a center of geometric asymmetry present in a compound of the invention, or in any chemical structure illustrated herein, is not specified, the structure is intended to encompass the trans geometric isomer, the cis geometric isomer, and all mixtures thereof. Likewise, all tautomeric forms are also included whether such tautomers exist in equilibrium or predominately in one form.

It is to be understood that the references herein to compounds of formula (I) and salts thereof covers the compounds of formula (I) as free acids or free bases, or as salts thereof, for example as pharmaceutically acceptable salts thereof. Thus, in one embodiment, the invention is directed to a compound of formula (I) as the free acid or free base. In another embodiment, the invention is directed to a compound of formula (I) or a salt thereof. In a further embodiment, the invention is directed to a compound of formula (I) or a pharmaceutically acceptable salt thereof.

The skilled artisan will appreciate that pharmaceutically acceptable salts of the compounds according to formula (I) may be prepared. Indeed, in certain embodiments of the invention, pharmaceutically acceptable salts of the compounds according to formula (I) may be preferred over the respective free base or free acid because such salts may impart greater stability or solubility to the molecule thereby facilitating formulation into a dosage form.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form, or a non-pharmaceutically acceptable salt, with a suitable base or acid, respectively.

Salts and solvates having non-pharmaceutically acceptable counter-ions or associated solvents are within the scope of the present invention, for example, for use as intermediates in the preparation of other compounds of formula (I) and their pharmaceutically acceptable salts. Thus one embodiment of the invention embraces compounds of formula (I) and salts thereof.

In certain embodiments, compounds according to formula (I) may contain an acidic functional group. Suitable pharmaceutically-acceptable salts include salts of such acidic functional groups. Representative salts include pharmaceutically acceptable metal salts such as sodium, potassium, lithium, calcium, magnesium, aluminum, and zinc salts; carbonates and bicarbonates of a pharmaceutically acceptable metal cation such as sodium, potassium, lithium, calcium, magnesium, aluminum, and zinc; pharmaceutically acceptable organic primary, secondary, and tertiary amines including aliphatic amines, aromatic amines, aliphatic diamines, and hydroxy alkylamines such as methylamine, ethylamine, 2-hydroxyethylamine, diethylamine, TEA, ethylenediamine, ethanolamine, diethanolamine, and cyclohexylamine.

In certain embodiments, compounds according to formula (I) may contain a basic functional group and are therefore capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methyl benzoate, dinitrobenzoate, hydroxybenzoate, methoxybenzoate, naphthoate, hydroxynaphthoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

### Compound Preparation

The compounds of the invention may be made by a variety of methods, including standard chemistry. Any previously defined variable will continue to have the previously defined meaning unless otherwise indicated. An illustrative general synthetic method is set out in Scheme 1 below and then specific compounds of the invention are prepared in the Examples section.

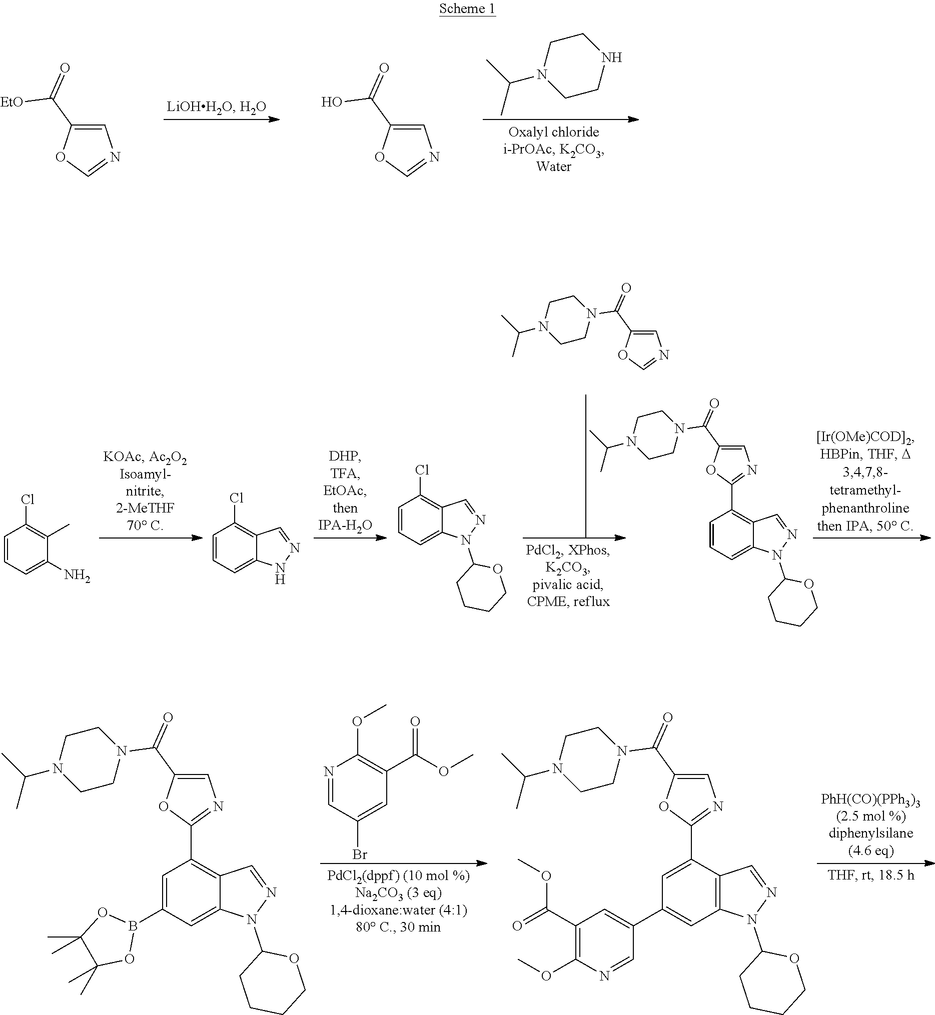

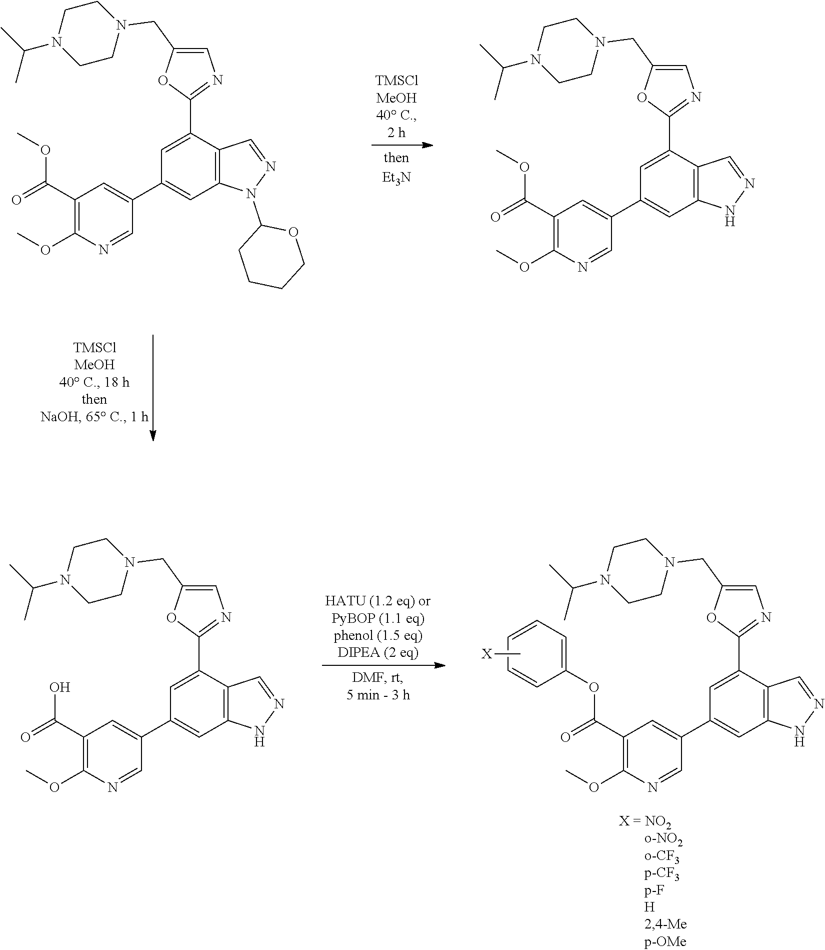

Thus, in one embodiment the invention provides a process for preparing a compound of formula (I) or a salt thereof comprising reacting a compound of formula (II) or a salt thereof

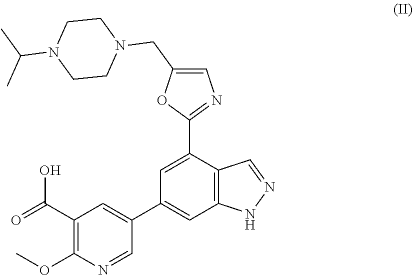 [SMILES: COc1ncc(-c2cc(-c3ncc(CN4CCN(C(C)C)CC4)o3)c3cn[nH]c3c2)cc1C(=O)O]

with a compound of formula (III) or a salt thereof

R⁶—OH (III)

wherein R⁶ is C₁₋₆alkyl is optionally substituted by —CF₃, or phenyl optionally substituted by one or two substituents independently selected from C₁₋₆alkyl, C₁₋₆alkoxy, halogen, —CN, —CF₃, —CO₂R², —CO₂NHR³, —NR⁴R⁵, —NO₂ and —SF₅.

### Methods of Use

The compounds of the invention are inhibitors of kinase activity, in particular PI3-kinase activity. Compounds which are PI3-kinase inhibitors may be useful in the treatment of disorders wherein the underlying pathology is (at least in part) attributable to inappropriate PI3-kinase activity, such as asthma and chronic obstructive pulmonary disease (COPD). “Inappropriate PI3-kinase activity” refers to any PI3-kinase activity that deviates from the normal PI3-kinase activity expected in a particular patient. Inappropriate PI3-kinase may take the form of, for instance, an abnormal increase in activity, or an aberration in the timing and or control of PI3-kinase activity. Such inappropriate activity may result then, for example, from overexpression or mutation of the PI3-kinase leading to inappropriate or uncontrolled activation. Accordingly, in another aspect the invention is directed to methods of treating such disorders.

Such disorders include respiratory diseases including asthma, chronic obstructive pulmonary disease (COPD) and idiopathic pulmonary fibrosis (IPF); ciliopathy including primary ciliary dyskinesia, polycystic liver disease and nephronophthisis; bacterial infections including bacterial respiratory tract infections, for example infections by S. Pneumoniae, H. Influenzae, M. Catarrhalis and/or mycobacteria such as Mycobacterium tuberculosis, and bacterial exacerbations of respiratory conditions and lung damage such as asthma, COPD and cystic fibrosis; viral infections including viral respiratory tract infections, for example infections by influenza, rhinovirus, respiratory syncytial virus (RSV), human parainfluenza virus (HPIV), adenovirus and/or coronavirus, and viral exacerbation of respiratory conditions and lung damage such as asthma, COPD and cystic fibrosis; other non-viral respiratory infections including aspergillosis and leishmaniasis; allergic diseases including allergic rhinitis, atopic dermatitis and psoriasis; autoimmune diseases including ankylosing spondylitis, Churg-Strauss syndrome, Crohn's disease, Glomerulonephritis, Henoch-Schonlein purpura, idiopathic thrombocytopenic purpura (ITP), interstitial cystitis, pemphigus, primary sclerosing cholangitis, psoriasis, rheumatoid arthritis, sarcoidosis, Sjogren's syndrome, Type 1 diabetes, ulcerative colitis, vasculitis and Wegener's granulomatosis; inflammatory disorders including inflammatory bowel disease; diabetes; cardiovascular diseases including thrombosis, atherosclerosis and hypertension; hematologic malignancies; neurodegenerative diseases; pancreatitis; multiorgan failure; kidney diseases; platelet aggregation; cancer; sperm motility; transplantation rejection; graft rejection; lung injuries; pain including pain associated with rheumatoid arthritis or osteoarthritis, back pain, general inflammatory pain, post hepatic neuralgia, diabetic neuropathy, inflammatory neuropathic pain (trauma), trigeminal neuralgia and Central pain; fibrotic diseases; depression; and psychotic disorders including schizophrenia.

Such fibrotic diseases may include idiopathic pulmonary fibrosis, interstitial lung diseases, non-specific interstitial pneumonia (NSIP), usual interstitial pneumonia (UIP), endomyocardial fibrosis, mediastinal fibrosis, myelofibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, renal fibrosis, hypertrophic cardiomyopathy (HCM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, and fibro-proliferative disorders.

In one embodiment, the disorder is asthma. In a further embodiment, the disorder is COPD.

Within the context of the present invention, the following terms describing the indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Within the context of the present invention, the term “psychotic disorder” includes Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

Within the context of the present invention, the term “depression” includes depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The methods of treatment of the invention comprise administering a safe and effective amount of a compound of formula (I) or a pharmaceutically acceptable salt thereof to a patient in need thereof.

Individual embodiments of the invention include methods of treating any one of the above-mentioned disorders by administering a safe and effective amount of a compound of formula (I) or a pharmaceutically acceptable salt thereof to a patient in need thereof.

As used herein, “treat” in reference to a disorder means: (1) to ameliorate or prevent the disorder or one or more of the biological manifestations of the disorder, (2) to interfere with (a) one or more points in the biological cascade that leads to or is responsible for the disorder or (b) one or more of the biological manifestations of the disorder, (3) to alleviate one or more of the symptoms or effects associated with the disorder, or (4) to slow the progression of the disorder or one or more of the biological manifestations of the disorder.

As indicated above, “treatment” of a disorder includes prevention of the disorder. The skilled artisan will appreciate that “prevention” is not an absolute term. In medicine, “prevention” is understood to refer to the prophylactic administration of a drug to substantially diminish the likelihood or severity of a disorder or biological manifestation thereof, or to delay the onset of such disorder or biological manifestation thereof. In one embodiment, the methods of the invention are directed to treating a disorder. In another embodiment, the methods of the invention are directed to preventing a disorder.

As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the disorder being treated; the severity of the disorder being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human (including adults and children) or other animal. In one embodiment, “patient” refers to a human.

The compounds of formula (I) or pharmaceutically acceptable salts thereof may be administered by any suitable route of administration, including both systemic administration and topical administration. Systemic administration includes oral administration, parenteral administration, transdermal administration and rectal administration. Parenteral administration refers to routes of administration other than enteral or transdermal, and is typically by injection or infusion. Parenteral administration includes intravenous, intramuscular, and subcutaneous injection or infusion. Topical administration includes application to the skin as well as intraocular, otic, intravaginal, inhaled and intranasal administration. Inhalation refers to administration into the patient's lungs whether inhaled through the mouth or through the nasal passages. In one embodiment, the compounds of formula (I) or pharmaceutically acceptable salts thereof may be administered orally. In another embodiment, the compounds of formula (I) or pharmaceutically acceptable salts thereof may be administered by inhalation. In a further embodiment, the compounds of formula (I) or pharmaceutically acceptable salts thereof may be administered intranasally.

The compounds of formula (I) or pharmaceutically acceptable salts thereof may be administered once or according to a dosing regimen wherein a number of doses are administered at varying intervals of time for a given period of time. For example, doses may be administered one, two, three, or four times per day. In one embodiment, a dose is administered once per day. In a further embodiment, a dose is administered twice per day. Doses may be administered until the desired therapeutic effect is achieved or indefinitely to maintain the desired therapeutic effect. Suitable dosing regimens for a compound of formula (I) or a pharmaceutically acceptable salt thereof depend on the pharmacokinetic properties of that compound, such as absorption, distribution, and half-life, which can be determined by the skilled artisan. In addition, suitable dosing regimens, including the duration such regimens are administered, for a compound of formula (I) or a pharmaceutically acceptable salt thereof depend on the disorder being treated, the severity of the disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical daily dosages for oral administration range from 0.001 mg to 50 mg per kg of total body weight, for example from 1 mg to 10 mg per kg of total body weight. For example, daily dosages for oral administration may be from 0.5 mg to 2 g per patient, such as 10 mg to 1 g per patient.

Additionally, the compounds of formula (I) may be administered as prodrugs. As used herein, a “prodrug” of a compound of formula (I) is a functional derivative of the compound which, upon administration to a patient, eventually liberates the compound of formula (I) in vivo. Administration of a compound of formula (I) as a prodrug may enable the skilled artisan to do one or more of the following: (a) modify the onset of the activity of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleavable in vivo. Such modifications, which include the preparation of phosphates, amides, esters, thioesters, carbonates, and carbamates, are well known to those skilled in the art.

In one aspect, the invention thus provides a method of treating a disorder mediated by inappropriate PI3-kinase activity comprising administering a safe and effective amount of a compound of formula (I) or a pharmaceutically acceptable salt thereof to a patient in need thereof.

In one embodiment, the disorder mediated by inappropriate PI3-kinase activity is selected from the group consisting of respiratory diseases (including asthma, chronic obstructive pulmonary disease (COPD) and idiopathic pulmonary fibrosis (IPF)); ciliopathy (including primary ciliary dyskinesia, polycystic liver disease and nephronophthisis); bacterial infections (including bacterial respiratory tract infections, for example infections by S. Pneumoniae, H. Influenzae, M. Catarrhalis and/or mycobacteria such as Mycobacterium tuberculosis) and bacterial exacerbations of respiratory conditions and lung damage (such as asthma, COPD and cystic fibrosis); viral infections (including viral respiratory tract infections, for example infections by influenza, rhinovirus, respiratory syncytial virus (RSV), human parainfluenza virus (HPIV), adenovirus and/or coronavirus) and viral exacerbation of respiratory conditions and lung damage (such as asthma, COPD and cystic fibrosis); other non-viral respiratory infections (including aspergillosis and leishmaniasis); allergic diseases (including allergic rhinitis, atopic dermatitis and psoriasis); autoimmune diseases (including ankylosing spondylitis, Churg-Strauss syndrome, Crohn's disease, Glomerulonephritis, Henoch-Schonlein purpura, idiopathic thrombocytopenic purpura (ITP), interstitial cystitis, pemphigus, primary sclerosing cholangitis, psoriasis, rheumatoid arthritis, sarcoidosis, Sjogren's syndrome, Type 1 diabetes, ulcerative colitis, vasculitis and Wegener's granulomatosis); inflammatory disorders (including inflammatory bowel disease); diabetes; cardiovascular diseases (including thrombosis, atherosclerosis and hypertension); hematologic malignancies; neurodegenerative diseases; pancreatitis; multiorgan failure; kidney diseases; platelet aggregation; cancer; sperm motility; transplantation rejection; graft rejection; lung injuries; pain (including pain associated with rheumatoid arthritis or osteoarthritis, back pain, general inflammatory pain, post hepatic neuralgia, diabetic neuropathy, inflammatory neuropathic pain (trauma), trigeminal neuralgia and Central pain); fibrotic diseases; depression; and psychotic disorders (including schizophrenia).

In one embodiment, the disorder mediated by inappropriate PI3-kinase activity is a respiratory disease. In another embodiment, the disorder mediated by inappropriate PI3-kinase activity is asthma. In a further embodiment, the disorder mediated by inappropriate PI3-kinase activity is chronic obstructive pulmonary disease (COPD).

In one aspect, the invention provides a compound of formula (I) or a pharmaceutically acceptable salt thereof for use in medical therapy.

In another aspect, the invention provides a compound of formula (I) or a pharmaceutically acceptable salt thereof for use in the treatment of a disorder mediated by inappropriate PI3-kinase activity.

In a further aspect, the invention provides the use of a compound of formula (I) or a pharmaceutically acceptable salt thereof in the manufacture of a medicament for use in the treatment of a disorder mediated by inappropriate PI3-kinase activity.

A number of different genetic variants in PI3Kδ have been observed (Jou et al., International Journal of Immunogenetics, 2006, 33, 361 to 369). One mutation (c.3061G>A, corresponding to m.3256G>A in the mRNA wherein the nucleotide number is based on the sequence data on GenBank: NM_005026) observed in a highly conserved position in the domain responsible for catalytic function results in a glutamic acid to lysine substitution (E1021K). It is believed that this mutation may result in patients being particularly susceptible to developing respiratory infections and/or exacerbations of respiratory infections, and damage to the airway wall, large and small airways, and lung parenchyma (Angulo et al., Science DOI: 10.1125/science. 1243292). Other gain of function mutations identified in the PIK3CD gene and leading to immune deficiencies include the amino acid residue substitution N334K or E525K (Lucas et al. Nat. Immunol. (2014) 15 p. 88-97). Mutations leading to aberrant splicing of PIK3R1 exon 10 and truncation of the p85a protein result in elevated PI3Kδ activity and to symptoms similar to the gain of function mutations in the PIK3CD gene (Deau et al. J. Clin. Invest. (2014) 124(9) p. 3923-8).

Thus, in one aspect, the invention thus provides a method of treating or preventing a respiratory infection, treating airway damage, and/or preventing airway injury in a patient with a PI3Kδ mutation, or increased PI3Kδ expression or activity, comprising administering a safe and effective amount of a compound of formula (I) or a pharmaceutically acceptable salt thereof to a patient in need thereof.

In one embodiment, the invention provides a compound of formula (I) or a pharmaceutically acceptable salt thereof for use in the treatment or prevention of a respiratory infection, the treatment of airway damage, and/or the prevention of airway injury in a patient with a PI3Kδ mutation, or increased PI3Kδ expression or activity.

In another embodiment, the invention provides the use of a compound of formula (I) or a pharmaceutically acceptable salt thereof in the manufacture of a medicament for use in the treatment or prevention of a respiratory infection, the treatment of airway damage, and/or the prevention of airway injury in a patient with a PI3Kδ mutation, or increased PI3Kδ expression or activity.

In another embodiment, the present invention provides a compound of formula (I) or a pharmaceutically acceptable salt thereof for use in the treatment or prevention of a respiratory infection, the treatment of airway damage, and/or the prevention of airway injury in a patient, comprising:
a) assaying a sample from the patient,
b) determining if the patient has a PI3Kδ mutation, or increased PI3Kδ expression or activity, and
c) administering a therapeutically effective amount of a compound of formula (I) or a pharmaceutically acceptable salt thereof to the patient if they have a PI3Kδ mutation, or increased PI3Kδ expression or activity.

In another embodiment, the invention provides a compound of formula (I) or a pharmaceutically acceptable salt thereof for use in the treatment or prevention of a respiratory infection, the treatment of airway damage, and/or the prevention of airway injury in a patient classified as a responder, wherein a responder is characterised by the presence of a PI3Kδ mutation, or increased PI3Kδ expression or activity.

In another embodiment, the invention provides use of a compound of formula (I) or a pharmaceutically acceptable salt thereof in the manufacture of a medicament for use in the treatment or prevention of a respiratory infection, the treatment of airway damage, and/or the prevention of airway injury in a patient classified as a responder, wherein a responder is characterised by the presence of a PI3Kδ mutation, or increased PI3Kδ expression or activity.

In a further embodiment, the invention provides a method of evaluating therapy with a compound of formula (I) or a pharmaceutically acceptable salt thereof, comprising:
a) obtaining a sample from the patient,
b) testing for a PI3Kδ mutation, or increased PI3Kδ expression or activity, and
c) determining if the patient should undergo therapy with a compound of formula (I) or a pharmaceutically acceptable salt thereof if a PI3Kδ mutation, or increased PI3Kδ expression or activity, is present.

Such respiratory infections may be the result of bacterial infections including, for example, infections by S. Pneumoniae, H. Influenzae, M. Catarrhalis and/or mycobacteria such as Mycobacterium tuberculosis; viral infections including, for example, infections by influenza, rhinovirus, respiratory syncytial virus (RSV), human parainfluenza virus (HPIV), adenovirus and/or coronavirus; and other non-viral respiratory infections including aspergillosis and/or leishmaniasis. In one embodiment, patients with a PI3Kδ mutation may be particularly susceptible to developing respiratory infections and/or exacerbations of respiratory infections as a result of bacterial infections by S. Pneumoniae, H. Influenzae, and/or M. Catarrhalis.

As used herein, the term “airway damage” refers to damage to the airway wall, large and small airways, and/or lung parenchyma which is present at the time a patient commences treatment. Airway damage, such as inflammation, scarring and/or remodelling, may be caused by, for example, repeated respiratory infections in a patient with a PI3Kδ mutation.

As used herein, the term “airway injury” refers to damage, or further damage, to the airway wall, large and small airways, and/or lung parenchyma which may develop in a patient if treatment does not occur.

As used herein, the term “responder” means someone who is identified (using a particular test or method) to be more likely to derive benefit in response to treatment (e.g. positive response to drug, reduction in adverse events, etc.). It is understood that not all people who have been identified as a responder will necessarily derive benefit, but as a patient class, they are more likely to do so. For example, it may be that out of the total untested diseased population, approximately 80% of that population derive benefit from a drug, but out of the group of “responders” (i.e. those individuals who have been tested, and identified as a responder according to the set criteria) approximately 99% will derive benefit.

As used herein, the term “evaluating therapy” means determining whether therapy with a compound of formula (I), or a pharmaceutically acceptable salt thereof, would be beneficial to a patient.

Patients with a PI3Kδ mutation may be particularly susceptible to an exacerbation of a respiratory infection. As used herein, the term “exacerbation of a respiratory infection” refers to a respiratory infection characterised by the worsening of an underlying persistent respiratory infection, including bacterial infections, viral infections and/or other non-viral respiratory infections. In one embodiment, the present invention thus provides a method of treating or preventing an exacerbation of a respiratory infection in a patient with a PI3Kδ mutation comprising administering a safe and effective amount of a compound of formula (I) or a pharmaceutically acceptable salt thereof to a patient in need thereof.

In one embodiment, the PI3Kδ mutation results in the substitution of glutamic acid for lysine. In another embodiment, the PI3Kδ mutation results in the substitution of glutamic acid for lysine at codon 1021 (E1021K).

In one embodiment, the PI3Kδ mutation results in a single base-pair missense mutation m.3256G>A in the mRNA (wherein the nucleotide number is based on the sequence data on GenBank: NM_005026).

In one embodiment, the PI3Kδ mutation is c.3061G>A.

The present inventors believe that by modifying selective reversible PI3Kδ inhibitors with carefully positioned electrophilic moieties, selective irreversible PI3Kδ inhibition may be achieved. Accordingly, in one embodiment, the present invention provides a selective irreversible PI3Kδ inhibitor comprising the moiety R¹OCO— wherein R¹ is phenyl optionally substituted by one or two substituents independently selected from C₁₋₆alkyl, C₁₋₆alkoxy, halogen, —CN, —CF₃, —CO₂R², —CO₂NHR³, —NR⁴R⁵, —NO₂ and —SF₅.

### Compositions

The compounds of formula (I) and pharmaceutically acceptable salts thereof will normally, but not necessarily, be formulated into pharmaceutical compositions prior to administration to a patient.

Accordingly, in one aspect the invention is directed to pharmaceutical compositions comprising a compound of formula (I) or a pharmaceutically acceptable salt thereof and one or more pharmaceutically acceptable excipients.

In another aspect the invention is directed to pharmaceutical compositions comprising 0.05 to 1000 mg of a compound of formula (I) or a pharmaceutically acceptable salt thereof and 0.1 to 2 g of one or more pharmaceutically acceptable excipients.

In a further aspect the invention is directed to a pharmaceutical composition for the treatment or prophylaxis of a disorder mediated by inappropriate PI3-kinase activity comprising a compound of formula (I) or a pharmaceutically acceptable salt thereof.

The pharmaceutical compositions of the invention may be prepared and packaged in bulk form wherein a safe and effective amount of a compound of formula (I) or a pharmaceutically acceptable salt thereof can be extracted and then given to the patient such as with powders or syrups. Alternatively, the pharmaceutical compositions of the invention may be prepared and packaged in unit dosage form wherein each physically discrete unit contains a compound of formula (I) or a pharmaceutically acceptable salt thereof. When prepared in unit dosage form, the pharmaceutical compositions of the invention typically may contain, for example, from 0.5 mg to 1 g, or from 1 mg to 700 mg, or from 5 mg to 100 mg of a compound of formula (I) or a pharmaceutically acceptable salt thereof.

The pharmaceutical compositions of the invention typically contain one compound of formula (I) or a pharmaceutically acceptable salt thereof.

As used herein, “pharmaceutically acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of formula (I) or a pharmaceutically acceptable salt thereof when administered to a patient and interactions which would result in pharmaceutical compositions that are not pharmaceutically acceptable are avoided. In addition, each excipient must of course be pharmaceutically-acceptable eg of sufficiently high purity.

The compound of formula (I) or a pharmaceutically acceptable salt thereof and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of formula (I) or pharmaceutically acceptable salts thereof once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other excipients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

Accordingly, in another aspect the invention is directed to process for the preparation of a pharmaceutical composition comprising a compound of formula (I) or a pharmaceutically acceptable salt thereof and one or more pharmaceutically acceptable excipients which comprises mixing the ingredients. A pharmaceutical composition comprising a compound of formula (I) or a pharmaceutically acceptable salt thereof may be prepared by, for example, admixture at ambient temperature and atmospheric pressure.

In one embodiment, the compounds of formula (I) or pharmaceutically acceptable salts thereof will be formulated for oral administration. In another embodiment, the compounds of formula (I) or pharmaceutically acceptable salts thereof will be formulated for inhaled administration. In a further embodiment, the compounds of formula (I) or pharmaceutically acceptable salts thereof will be formulated for intranasal administration.

In one aspect, the invention is directed to a solid oral dosage form such as a tablet or capsule comprising a safe and effective amount of a compound of formula (I) or a pharmaceutically acceptable salt thereof and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

The compounds of formula (I) or pharmaceutically acceptable salts thereof may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds of formula (I) or pharmaceutically acceptable salts thereof may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

In another aspect, the invention is directed to a liquid oral dosage form. Oral liquids such as solution, syrups and elixirs can be prepared in dosage unit form so that a given quantity contains a predetermined amount of a compound of formula (I) or a pharmaceutically acceptable salt thereof. Syrups can be prepared by dissolving the compound of formula (I) or a pharmaceutically acceptable salt thereof in a suitably flavored aqueous solution, while elixirs are prepared through the use of a non-toxic alcoholic vehicle. Suspensions can be formulated by dispersing the compound of formula (I) or a pharmaceutically acceptable salt thereof in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another aspect, the invention is directed to a dosage form adapted for administration to a patient by inhalation, for example as a dry powder, an aerosol, a suspension, or a solution composition. In one embodiment, the invention is directed to a dosage form adapted for administration to a patient by inhalation as a dry powder. In a further embodiment, the invention is directed to a dosage form adapted for administration to a patient by inhalation via a nebulizer.

Dry powder compositions for delivery to the lung by inhalation typically comprise a compound of formula (I) or a pharmaceutically acceptable salt thereof as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides. The finely divided powder may be prepared by, for example, micronisation and milling. Generally, the size-reduced (eg micronised) compound can be defined by a D₅₀ value of about 1 to about 10 microns (for example as measured using laser diffraction).

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of the compound of formula (I) or a pharmaceutically acceptable salt thereof.

Aerosols may be formed by suspending or dissolving a compound of formula (I) or a pharmaceutically acceptable salt thereof in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of formula (I) or a pharmaceutically acceptable salt thereof will typically be administered to a patient via a metered dose inhaler (MDI). Such devices are known to those skilled in the art.

The aerosol may contain additional pharmaceutically-acceptable excipients typically used with MDIs such as surfactants, lubricants, cosolvents and other excipients to improve the physical stability of the formulation, to improve valve performance, to improve solubility, or to improve taste.

There is thus provided as a further aspect of the invention a pharmaceutical aerosol formulation comprising a compound of formula (I) or a pharmaceutically acceptable salt thereof and a fluorocarbon or hydrogen-containing chlorofluorocarbon as propellant, optionally in combination with a surfactant and/or a cosolvent.

According to another aspect of the invention, there is provided a pharmaceutical aerosol formulation wherein the propellant is selected from 1,1,1,2-tetrafluoroethane, 1,1,1,2,3,3,3-heptafluoro-n-propane and mixtures thereof.

The formulations of the invention may be buffered by the addition of suitable buffering agents.

Capsules and cartridges for use in an inhaler or insufflator, of for example gelatine, may be formulated containing a powder mix for inhalation of a compound of formula (I) or a pharmaceutically acceptable salt thereof and a suitable powder base such as lactose or starch. Each capsule or cartridge may generally contain from 20 μg to 10 mg of the compound of formula (I) or pharmaceutically acceptable salt thereof. Alternatively, the compound of formula (I) or pharmaceutically acceptable salt thereof may be presented without excipients such as lactose.

The proportion of the active compound of formula (I) or pharmaceutically acceptable salt thereof in the local compositions according to the invention depends on the precise type of formulation to be prepared but will generally be within the range of from 0.001 to 10% by weight. Generally, for most types of preparations, the proportion used will be within the range of from 0.005 to 1%, for example from 0.01 to 0.5%. However, in powders for inhalation or insufflation the proportion used will normally be within the range of from 0.1 to 5%.

Aerosol formulations are preferably arranged so that each metered dose or “puff” of aerosol contains from 20 μg to 10 mg, preferably from 20 μg to 2000 μg, more preferably from about 20 μg to 500 μg of a compound of formula (I). Administration may be once daily or several times daily, for example 2, 3, 4 or 8 times, giving for example 1, 2 or 3 doses each time. The overall daily dose with an aerosol will be within the range from 100 μg to 10 mg, preferably from 200 μg to 2000 μg. The overall daily dose and the metered dose delivered by capsules and cartridges in an inhaler or insufflator will generally be double that delivered with aerosol formulations.

In the case of suspension aerosol formulations, the particle size of the particulate (e.g., micronised) drug should be such as to permit inhalation of substantially all the drug into the lungs upon administration of the aerosol formulation and will thus be less than 100 microns, desirably less than 20 microns, and in particular in the range of from 1 to 10 microns, such as from 1 to 5 microns, more preferably from 2 to 3 microns.

The formulations of the invention may be prepared by dispersal or dissolution of the medicament and a compound of formula (I) or a pharmaceutically acceptable salt thereof in the selected propellant in an appropriate container, for example, with the aid of sonication or a high-shear mixer. The process is desirably carried out under controlled humidity conditions.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The stability of the suspension aerosol formulations according to the invention may be measured by conventional techniques, for example, by measuring flocculation size distribution using a back light scattering instrument or by measuring particle size distribution by cascade impaction or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above.

The term “metered dose inhaler” or MDI means a unit comprising a can, a secured cap covering the can and a formulation metering valve situated in the cap. MDI system includes a suitable channelling device. Suitable channelling devices comprise for example, a valve actuator and a cylindrical or cone-like passage through which medicament may be delivered from the filled canister via the metering valve to the nose or mouth of a patient such as a mouthpiece actuator.

MDI canisters generally comprise a container capable of withstanding the vapour pressure of the propellant used such as a plastic or plastic-coated glass bottle or preferably a metal can, for example, aluminium or an alloy thereof which may optionally be anodised, lacquer-coated and/or plastic-coated (for example incorporated herein by reference WO96/32099 wherein part or all of the internal surfaces are coated with one or more fluorocarbon polymers optionally in combination with one or more non-fluorocarbon polymers), which container is closed with a metering valve. The cap may be secured onto the can via ultrasonic welding, screw fitting or crimping. MDIs taught herein may be prepared by methods of the art (e.g. see Byron, above and WO96/32099). Preferably the canister is fitted with a cap assembly, wherein a drug-metering valve is situated in the cap, and said cap is crimped in place.

In one embodiment of the invention the metallic internal surface of the can is coated with a fluoropolymer, more preferably blended with a non-fluoropolymer. In another embodiment of the invention the metallic internal surface of the can is coated with a polymer blend of polytetrafluoroethylene (PTFE) and polyethersulfone (PES). In a further embodiment of the invention the whole of the metallic internal surface of the can is coated with a polymer blend of polytetrafluoroethylene (PTFE) and polyethersulfone (PES).

The metering valves are designed to deliver a metered amount of the formulation per actuation and incorporate a gasket to prevent leakage of propellant through the valve. The gasket may comprise any suitable elastomeric material such as, for example, low density polyethylene, chlorobutyl, bromobutyl, EPDM, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK357) and 3M-Neotechnic Ltd, UK (e.g. Spraymiser™).

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; and 6,679,374, as well as dose counter units such as, but not limited to, those described in U.S. Pat. Nos. 6,360,739 and 6,431,168.

Conventional bulk manufacturing methods and machinery well known to those skilled in the art of pharmaceutical aerosol manufacture may be employed for the preparation of large-scale batches for the commercial production of filled canisters. Thus, for example, in one bulk manufacturing method for preparing suspension aerosol formulations a metering valve is crimped onto an aluminium can to form an empty canister. The particulate medicament is added to a charge vessel and liquefied propellant together with the optional excipients is pressure filled through the charge vessel into a manufacturing vessel. The drug suspension is mixed before recirculation to a filling machine and an aliquot of the drug suspension is then filled through the metering valve into the canister. In one example bulk manufacturing method for preparing solution aerosol formulations a metering valve is crimped onto an aluminium can to form an empty canister. The liquefied propellant together with the optional excipients and the dissolved medicament is pressure filled through the charge vessel into a manufacturing vessel.

In an alternative process, an aliquot of the liquefied formulation is added to an open canister under conditions which are sufficiently cold to ensure the formulation does not vaporise, and then a metering valve crimped onto the canister.

Typically, in batches prepared for pharmaceutical use, each filled canister is check-weighed, coded with a batch number and packed into a tray for storage before release testing.

Suspensions and solutions comprising a compound of formula (I) or a pharmaceutically acceptable salt thereof may also be administered to a patient via a nebulizer. The solvent or suspension agent utilized for nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically-acceptable excipients may be added to the suspension or solution. The compound of formula (I) or pharmaceutically acceptable salt thereof may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of formula (I) or pharmaceutically acceptable salt thereof. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

In a further aspect, the invention is directed to a dosage form adapted for intranasal administration.

Formulations for administration to the nose may include pressurised aerosol formulations and aqueous formulations administered to the nose by pressurised pump. Formulations which are non-pressurised and adapted to be administered topically to the nasal cavity are of particular interest. Suitable formulations contain water as the diluent or carrier for this purpose. Aqueous formulations for administration to the lung or nose may be provided with conventional excipients such as buffering agents, tonicity modifying agents and the like. Aqueous formulations may also be administered to the nose by nebulisation.

The compounds of formula (I) or pharmaceutically acceptable salts thereof may be formulated as a fluid formulation for delivery from a fluid dispenser, for example a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO05/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. In one embodiment, the fluid dispenser is of the general type illustrated in FIGS. 30-40 of WO05/044354.

Pharmaceutical compositions adapted for intranasal administration wherein the carrier is a solid include a coarse powder having a particle size for example in the range 20 to 500 microns which is administered by rapid inhalation through the nasal passage from a container of the powder held close up to the nose. Suitable compositions wherein the carrier is a liquid, for administration as a nasal spray or as nasal drops, include aqueous or oil solutions of the compound of formula (I) or a pharmaceutically acceptable salt thereof.

Pharmaceutical compositions adapted for transdermal administration may be presented as discrete patches intended to remain in intimate contact with the epidermis of the patient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, ca rboxypolymethylene and cellulose derivatives, and/or glyceryl monostearate and/or non-ionic emulsifying agents.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents, suspending agents or preservatives.

Topical preparations may be administered by one or more applications per day to the affected area; over skin areas occlusive dressings may advantageously be used. Continuous or prolonged delivery may be achieved by an adhesive reservoir system.

For treatments of the eye or other external tissues, for example mouth and skin, the compositions may be applied as a topical ointment or cream. When formulated in an ointment, the compound of formula (I) or a pharmaceutically acceptable salt thereof may be employed with either a paraffinic or a water-miscible ointment base. Alternatively, the compound of formula (I) or pharmaceutically acceptable salt thereof may be formulated in a cream with an oil-in-water cream base or a water-in-oil base.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compound and pharmaceutical formulations according to the invention may be used in combination with or include one or more other therapeutic agents, for example selected from anti-inflammatory agents, anticholinergic agents, β₂-adrenoreceptor agonists, leukotriene antagonists (such as montelukast, zafirlukast or pranlukast), antiinfective agents, antihistamines, antigen immunotherapy, corticosteroids (such as fluticasone propionate, fluticasone furoate, beclomethasone diproprionate, budesonide, ciclesonide, mometasone furoate, triamcinolone or flunisolide), iNOS inhibitors, tryptase inhibitors, IKK2 inhibitors, p38 inhibitors, Syk inhibitors, elastase inhibitors, beta-2 integrin antagonists, adenosine ata agonists, chemokine antagonists such as CCR3 antagonists or CCR4 antagonists, mediator release inhibitors (such as sodium chromoglycate), 5-lipoxygenase inhibitors (zyflo), DP1 antagonists, DP2 antagonists, PDE4 inhibitors, PI3-kinase inhibitors, PI4-kinase inhibitors, ITK inhibitors, LP (lysophosphatidic) inhibitors, FLAP (5-lipoxygenase activating protein) inhibitors (such as sodium 3-(3-(tert-butylthio)-1-(4-(6-ethoxypyridin-3-yl)benzyl)-5-((5-methylpyridin-2-yl)methoxy)-1H-indol-2-yl)-2,2-dimethylpropanoate), DMARDs (disease-modifying anti-rheumatic drugs) (such as methotrexate, leflunomide or azathioprine), monoclonal antibody therapy (such as anti-TSLP, anti-IgE, anti-TNF, anti-IL-5, anti-IL-6, anti-IL-12 or anti-IL-1), receptor therapies (such as etanercept), and/or antigen non-specific immunotherapies (such as interferon or other cytokines/chemokines, cytokine/chemokine receptor modulators, cytokine agonists or antagonists, or TLR agonists).

The invention thus provides, in a further aspect, a combination comprising a compound of formula (I) or a pharmaceutically acceptable salt thereof together with one or more other therapeutically active agents, for example selected from an anti-inflammatory agent, an anticholinergic agent, a β₂-adrenoreceptor agonist, a leukotriene antagonist, an antiinfective agent, an antihistamine, antigen immunotherapy, a corticosteroid, an iNOS inhibitor, a tryptase inhibitor, an IKK2 inhibitor, a p38 inhibitor, a Syk inhibitor, an elastase inhibitor, a beta-2 integrin antagonist, an adenosine ata agonist, a chemokine antagonist, a mediator release inhibitor, a 5-lipoxygenase inhibitors, a DP1 antagonist, a DP2 antagonist, a PDE4 inhibitor, a PI3-kinase inhibitor, a PI4-kinase inhibitor, an ITK inhibitor, a LP (lysophosphatidic) inhibitor, a FLAP (5-lipoxygenase activating protein) inhibitor, a DMARD, monoclonal antibody therapy, receptor therapy, and/or antigen non-specific immunotherapy.

In one embodiment, the invention encompasses a method of treating a disorder mediated by inappropriate PI3-kinase activity comprising administering a safe and effective amount of a combination comprising a compound of formula (I) or a pharmaceutically acceptable salt thereof together with one or more therapeutically active agents.

Certain compounds of the invention may show selectivity for PI3Kδ over other PI3-kinases. The invention thus provides, in a further aspect, a combination comprising a compound of formula (I) or a pharmaceutically acceptable salt thereof which is selective for PI3Kδ together with a compound or pharmaceutically acceptable salt thereof which is selective for another PI3-kinase, for example PI3 Ky.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimise the activity and/or stability and/or physical characteristics, such as solubility, of the therapeutic ingredient. It will be clear also that, where appropriate, the therapeutic ingredients may be used in optically pure form.

In one embodiment, the invention encompasses a combination comprising a compound of formula (I) or a pharmaceutically acceptable salt thereof together with a β₂-adrenoreceptor agonist.

Examples of β₂-adrenoreceptor agonists include salmeterol (which may be a racemate or a single enantiomer such as the R-enantiomer), salbutamol (which may be a racemate or a single enantiomer such as the R-enantiomer), formoterol (which may be a racemate or a single duastereomer such as the R,R-diastereomer), salmefamol, fenoterol carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol. In one embodiment, long-acting β₂-adrenoreceptor agonists, for example, compounds which provide effective bronchodilation for about 12 hrs or longer, are preferred.

Other β₂-adrenoreceptor agonists include those described in WO 02/066422, WO 02/070490, WO 02/076933, WO 03/024439, WO 03/072539, WO 03/091204, WO 04/016578, WO 2004/022547, WO 2004/037807, WO 2004/037773, WO 2004/037768, WO 2004/039762, WO 2004/039766, WO01/42193 and WO03/042160.

Examples of β₂-adrenoreceptor agonists include:
3-(4-{[6-({(2R)-2-hydroxy-2-[4-hydroxy-3-(hydroxymethyl)phenyl]ethyl}amino) hexyl]oxy}butyl)benzenesulfonamide;
3-(3-{[7-({(2R)-2-hydroxy-2-[4-hydroxy-3-hydroxymethyl)phenyl]ethyl}amino)heptyl]oxy}propyl) benzenesulfonamide;
4-{(1R)-2-[(6-{2-[(2,6-dichlorobenzyl)oxy]ethoxy}hexyl)amino]-1-hydroxyethyl}-2-(hydroxymethyl) phenol;
4-{(1R)-2-[(6-{4-[3-(cyclopentylsulfonyl)phenyl]butoxy}hexyl)amino]-1-hydroxyethyl}-2-(hydroxymethyl)phenol;
N-[2-hydroxyl-5-[(1R)-1-hydroxy-2-[[2-4-[[(2R)-2-hydroxy-2-phenylethyl]amino]phenyl]-ethyl]amino]ethyl]phenyl]formamide;
N-2{2-[4-(3-phenyl-4-methoxyphenyl)aminophenyl]ethyl}-2-hydroxy-2-(8-hydroxy-2(1H)-quinolinon-5-yl)ethylamine; and
5-[(R)-2-(2-{4-[4-(2-amino-2-methyl-propoxy)-phenylamino]-phenyl}-ethylamino)-1-hydroxy-ethyl]-8-hydroxy-1H-quinolin-2-one.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

In one embodiment, the invention encompasses a combination comprising a compound of formula (I) or a pharmaceutically acceptable salt thereof together with a leukotriene antagonist. Suitable leukotriene antagonists include, for example, montelukast.

Suitable anti-inflammatory agents include corticosteroids. Suitable corticosteroids which may be used in combination with the compounds of formula (I) or pharmaceutically acceptable salts thereof are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methylcyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. Preferred corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid 5-fluoromethyl ester, 6α,9α-difluoro-17α-[(2-furanylcarbonyl)oxy]-11β-hydroxy-16α-methyl-3-oxo-androsta-1,4-diene-17β-carbothioic acid 5-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid 5-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid 5-fluoromethyl ester. In one embodiment the corticosteroid is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Examples of corticosteroids may include those described in WO2002/088167, WO2002/100879, WO2002/12265, WO2002/12266, WO2005/005451, WO2005/005452, WO2006/072599 and WO2006/072600.

Non-steroidal compounds having glucocorticoid agonism that may possess selectivity for transrepression over transactivation and that may be useful in combination therapy include those covered in the following patents: WO03/082827, WO98/54159, WO04/005229, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651 and WO03/08277. Further non-steroidal compounds are covered in: WO2006/000401, WO2006/000398 and WO2006/015870.

Examples of anti-inflammatory agents include non-steroidal anti-inflammatory drugs (NSAID's).

Examples of NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example montelukast), tryptase and elastase inhibitors, beta-2 integrin antagonists and adenosine receptor agonists or antagonists (e.g. adenosine 2a agonists), cytokine antagonists, or inhibitors of cytokine synthesis, or 5-lipoxygenase inhibitors.

In one embodiment, the invention provides the use of the compounds of formula (I) in combination with a phosphodiesterase 4 (PDE4) inhibitor, especially in the case of a formulation adapted for inhalation. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxy-phenyl)cyclohexan-1-one and cis-[4-cyano-4-(3-cyclopropylmethoxy-4-difluoromethoxy-phenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep. 1996; this patent and the compounds it discloses are incorporated herein in full by reference.

Other compounds include AWD-12-281 from Elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Further compounds are disclosed in the published international patent application WO04/024728 (Glaxo Group Ltd), WO04/056823 (Glaxo Group Ltd) and WO04/103998 (Glaxo Group Ltd) (e.g. Example 399 or 544 disclosed therein). Further compounds are also disclosed in WO2005/058892, WO2005/090348, WO2005/090353, and WO2005/090354, all in the name of Glaxo Group Limited.

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (for example, as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Additional compounds are disclosed in WO 2005/037280, WO 2005/046586 and WO 2005/104745, incorporated herein by reference. The present combinations include, but are not limited to:
(3-endo)-3-(2,2-di-2-thienylethenyl)-8,8-dimethyl-8-azoniabicyclo[3.2.1]octane iodide;
(3-endo)-3-(2-cyano-2,2-diphenylethyl)-8,8-dimethyl-8-azoniabicyclo[3.2.1]octane bromide;
4-[hydroxy(diphenyl)methyl]-1-{2-[(phenylmethyl)oxy]ethyl}-1-azoniabicyclo[2.2.2]octane bromide; and
(1R,5S)-3-(2-cyano-2,2-diphenylethyl)-8-methyl-8-{2-[(phenylmethyl)oxy]ethyl}-8-azoniabicyclo[3.2.1]octane bromide.

Other anticholinergic agents include compounds which are disclosed in U.S. patent application 60/487,981 including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methyl-benzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further anticholinergic agents include compounds which are disclosed in U.S. patent application 60/511,009 including, for example:
(endo)-3-(2-methoxy-2,2-di-thiophen-2-yl-ethyl)-8,8-dimethyl-8-azonia-bicyclo[3.2.1]octane iodide;
3-((endo)-8-methyl-8-aza-bicyclo[3.2.1]oct-3-yl)-2,2-diphenyl-propionitrile;
(endo)-8-methyl-3-(2,2,2-triphenyl-ethyl)-8-aza-bicyclo[3.2.1]octane;
3-((endo)-8-methyl-8-aza-bicyclo[3.2.1]oct-3-yl)-2,2-diphenyl-propionamide;
3-((endo)-8-methyl-8-aza-bicyclo[3.2.1]oct-3-yl)-2,2-diphenyl-propionic acid;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
3-((endo)-8-methyl-8-aza-bicyclo[3.2.1]oct-3-yl)-2,2-diphenyl-propan-1-ol;
N-benzyl-3-((endo)-8-methyl-8-aza-bicyclo[3.2.1]oct-3-yl)-2,2-diphenyl-propionamide;
(endo)-3-(2-carbamoyl-2,2-diphenyl-ethyl)-8,8-dimethyl-8-azonia-bicyclo[3.2.1]octane iodide;
1-benzyl-3-[3-((endo)-8-methyl-8-aza-bicyclo[3.2.1]oct-3-yl)-2,2-diphenyl-propyl]-urea;
1-ethyl-3-[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-acetamide;
N-[3-((endo)-8-methyl-8-aza-bicyclo[3.2.1]oct-3-yl)-2,2-diphenyl-propyl]-benzamide;
3-((endo)-8-methyl-8-aza-bicyclo[3.2.1]oct-3-yl)-2,2-di-thiophen-2-yl-propionitrile;
(endo)-3-(2-cyano-2,2-di-thiophen-2-yl-ethyl)-8,8-dimethyl-8-azonia-bicyclo[3.2.1]octane iodide;
N-[3-((endo)-8-methyl-8-aza-bicyclo[3.2.1]oct-3-yl)-2,2-diphenyl-propyl]-benzene-sulfonamide;
[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-methane-sulfonamide; and/or
(endo)-3-{2,2-diphenyl-3-[(1-phenyl-methanoyl)-amino]-propyl}-8,8-dimethyl-8-azonia-bicyclo[3.2.1]octane bromide.

Further compounds include:
(endo)-3-(2-methoxy-2,2-di-thiophen-2-yl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
(endo)-3-(2-carbamoyl-2,2-diphenyl-ethyl)-8,8-dimethyl-8-azonia-bicyclo[3.2.1]octane iodide;
(endo)-3-(2-cyano-2,2-di-thiophen-2-yl-ethyl)-8,8-dimethyl-8-azonia-bicyclo[3.2.1]octane iodide; and/or
(endo)-3-{2,2-diphenyl-3-[(1-phenyl-methanoyl)-amino]-propyl}-8,8-dimethyl-8-azonia-bicyclo[3.2.1]octane bromide.

In one embodiment the invention provides a combination comprising a compound of formula (I) or a pharmaceutically acceptable salt thereof together with an H1 antagonist. Examples of H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of formula (I) or a pharmaceutically acceptable salt thereof together with an H3 antagonist (and/or inverse agonist). Examples of H3 antagonists include, for example, those compounds disclosed in WO2004/035556 and in WO2006/045416. Other histamine receptor antagonists which may be used in combination with the compounds of the present invention include antagonists (and/or inverse agonists) of the H4 receptor, for example, the compounds disclosed in Jablonowski et al., J. Med. Chem. 46:3957-3960 (2003).

In one embodiment the invention provides a combination comprising a compound of formula (I) or a pharmaceutically acceptable salt thereof together with an anti-infective agent. The anti-infective agent may be an antibiotic, an antiviral or an antifungal. Examples of suitable antibiotics may include amoxicillin/clavulanate, flucloxacillin, cefalexin, cefixime, erythromycin, ciprofloxacin and tobramycin. Examples of suitable antivirals may include oseltamivir, zanamivir and ribavirin. Examples of suitable antifungals may include fluconazole and itraconazole.

In one embodiment the combination comprising a compound of formula (I) or a pharmaceutically acceptable salt thereof together with an anti-infective agent may be administered by inhalation. Examples of anti-infective agents particularly suitable for inhalation include those that may be inhaled or nebulized, for example, antibiotics such as tobramycin or ciprofloxacin, and antivirals such as zanamivir or ribavirin.

In one embodiment the invention provides a combination comprising a compound of formula (I) or a pharmaceutically acceptable salt thereof together with an anti-infective agent that has a compatible duration of action with the compound of formula (I). By the term “compatible duration of action” as used herein, is meant that the duration of action is such that both compounds may be administered to treat a particular patient, for example, they may be administered the same number of times each day such as once daily or 2, 3, 4 or 8 times.

The invention thus provides, in a further aspect, a combination comprising a compound of formula (I) or a pharmaceutically acceptable salt thereof together with a PDE4 inhibitor.

The invention thus provides, in a further aspect, a combination comprising a compound of formula (I) or a pharmaceutically acceptable salt thereof together with a β₂-adrenoreceptor agonist.

The invention thus provides, in a further aspect, a combination comprising a compound of formula (I) or a pharmaceutically acceptable salt thereof together with a leukotriene antagonist.

The invention thus provides, in a further aspect, a combination comprising a compound of formula (I) or a pharmaceutically acceptable salt thereof together with a corticosteroid.

The invention thus provides, in a further aspect, a combination comprising a compound of formula (I) or a pharmaceutically acceptable salt thereof together with a non-steroidal GR agonist.

The invention thus provides, in a further aspect, a combination comprising a compound of formula (I) or a pharmaceutically acceptable salt thereof together with an anticholinergic.

The invention thus provides, in a further aspect, a combination comprising a compound of formula (I) or a pharmaceutically acceptable salt thereof together with an antihistamine.

The invention thus provides, in a further aspect, a combination comprising a compound of formula (I) or a pharmaceutically acceptable salt thereof together with a PDE4 inhibitor and a β₂-adrenoreceptor agonist.

The invention thus provides, in a further aspect, a combination comprising a compound of formula (I) or a pharmaceutically acceptable salt thereof together with an anticholinergic and a PDE-4 inhibitor.

The invention thus provides, in a further aspect, a combination comprising a compound of formula (I) or a pharmaceutically acceptable salt thereof together with an anti-infective agent.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilled in the art.

The invention thus provides, in a further aspect, a pharmaceutical composition comprising a combination of a compound of formula (I) or a pharmaceutically acceptable salt thereof together with another therapeutically active agent.

The invention thus provides, in a further aspect, a pharmaceutical composition comprising a combination of a compound of formula (I) or a pharmaceutically acceptable salt thereof together with a PDE4 inhibitor.

The invention thus provides, in a further aspect, a pharmaceutical composition comprising a combination of a compound of formula (I) or a pharmaceutically acceptable salt thereof together with a β₂-adrenoreceptor agonist.

The invention thus provides, in a further aspect, a pharmaceutical composition comprising a combination of a compound of formula (I) or a pharmaceutically acceptable salt thereof together with a leukotriene antagonist.

The invention thus provides, in a further aspect, a pharmaceutical composition comprising a combination of a compound of formula (I) or a pharmaceutically acceptable salt thereof together with a corticosteroid.

The invention thus provides, in a further aspect, a pharmaceutical composition comprising a combination of a compound of formula (I) or a pharmaceutically acceptable salt thereof together with a non-steroidal GR agonist.

The invention thus provides, in a further aspect, a pharmaceutical composition comprising a combination of a compound of formula (I) or a pharmaceutically acceptable salt thereof together with an anticholinergic.

The invention thus provides, in a further aspect, a pharmaceutical composition comprising a combination of a compound of formula (I) or a pharmaceutically acceptable salt thereof together with an antihistamine.

The invention thus provides, in a further aspect, a pharmaceutical composition comprising a combination of a compound of formula (I) or a pharmaceutically acceptable salt thereof together with a PDE4 inhibitor and a 132-adrenoreceptor agonist.

The invention thus provides, in a further aspect, a pharmaceutical composition comprising a combination of a compound of formula (I) or a pharmaceutically acceptable salt thereof together with an anticholinergic and a PDE4 inhibitor.

The invention thus provides, in a further aspect, a pharmaceutical composition comprising a combination of a compound of formula (I) or a pharmaceutically acceptable salt thereof together with an anti-infective agent.

The invention will now be illustrated by way of the following non-limiting examples.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

The names of the Examples have been obtained using a compound naming programme which matches structure to name (e.g. ACD/Name Batch v 9.0).

When the name of a commercial supplier is given after the name of a compound or a reagent, this means that the compound is obtainable from a commercial supplier, such as the commercial supplier named. If not referenced herein the compound or reagent can be purchased from a standard supplier such as Sigma Aldrich, Lancaster, Fluorochem, TCI etc.

### General Methods

### Liquid Chromatography Mass Spectrometry (LCMS)

Reaction progress and final LCMS analyses were conducted using one of the three methods below.

### LCMS Method A

The liquid chromatography (LC) analysis was conducted on an Acquity UPLC CSH C18 column (50 mm×2.1 mm internal diameter, 1.7 μm packing diameter) at 40° C. using a 0.5 μL injection volume.

The solvents employed were:

A=0.1% v/v solution of formic acid in water.

B=0.1% v/v solution of formic acid in acetonitrile.

The gradient employed was:

| | Flow Rate/ | | |
|---|---|---|---|
| Time/min | mL · min⁻¹ | % A | % B |
| 0.00 | 1 | 97 | 3 |
| 1.50 | 1 | 5 | 95 |
| 1.90 | 1 | 5 | 95 |
| 2.00 | 1 | 97 | 3 |

The UV detection was a summed signal from a wavelength of 210 nm to 350 nm. Mass spectra were recorded on a Waters ZQ mass spectrometer using alternate-scan positive and negative electrospray ionisation (ES⁺ and ES⁻) with a scan range of 100 to 1000 amu, scan time of 0.27 s and an inter-scan delay of 0.10 s.

### LCMS Method B

The liquid chromatography (LC) analysis was conducted on an Acquity UPLC CSH C18 column (50 mm×2.1 mm internal diameter, 1.7 μm packing diameter) at 40° C. using a 0.5 μL injection volume.

The solvents employed were:

A=0.1% v/v solution of trifluoroacetic acid in water.

B=0.1% v/v solution of trifluoroacetic acid in acetonitrile.

The gradient employed was:

| | Flow Rate/ | | |
|---|---|---|---|
| Time/min | mL · min⁻¹ | % A | % B |
| 0.00 | 1 | 95 | 5 |
| 1.50 | 1 | 5 | 95 |
| 1.90 | 1 | 5 | 95 |
| 2.00 | 1 | 95 | 5 |

The UV detection was a summed signal from a wavelength of 210 nm to 350 nm. Mass spectra were recorded on a Waters ZQ mass spectrometer using positive electrospray ionisation (ES⁺) with a scan range of 100 to 1000 amu, scan time of 0.27 s and an inter-scan delay of 0.05 s.

### Mass Directed Automated Preparative HPLC (MDAP)

### Method A

Column: Xselect CSH C18 column (150 mm×30 mm i.d. 5 μm packing diameter) at ambient temperature.

The solvents employed were:

A=10 mM ammonium bicarbonate adjusted to pH 10 with ammonia in water.

B=MeCN.

Injection Volume: 1 mL

The DAD detection was 210 nm to 350 nm.

MS Conditions

MS: Waters ZQ

Ionisation mode: Alternate scan positive/negative Electrospray

Scan Range: 100 to 1000 AMU

Scan Time: 0.50 s

Inter scan Delay: 0.2 s

### Method B

Column: Xselect CSH C18 column (150 mm×30 mm i.d. 5 μm packing diameter) at ambient temperature.

The solvents employed were:

A=0.1% v/v solution of formic acid in water

B=0.1% v/v solution of formic acid in MeCN.

Injection Volume: 1 mL

The DAD detection was 210 nm to 350 nm.

MS Conditions

MS: Waters ZQ

Ionisation mode: Alternate scan positive/negative Electrospray

Scan Range: 100 to 1000 AMU

Scan Time: 0.50 s

Inter scan Delay: 0.2 s

### Method C

Column: Xselect CSH C18 column (150 mm×30 mm i.d. 5 μm packing diameter) at ambient temperature.

The solvents employed were:

A=10 mM ammonium bicarbonate in water adjusted to pH 10 with ammonia.

B=MeCN.

Injection Volume: 3 mL

The UV detection was for a signal wavelength at 254 nm.

MS Conditions

MS: Waters ZQ

Ionisation mode: Alternate scan positive/negative Electrospray

Scan Range: 100 to 1000 AMU

Scan Time: 0.50 s

Inter scan Delay: 0.2 s

### Method D

Column: Xselect CSH C18 column (150 mm×30 mm i.d. 5 μm packing diameter) at ambient temperature.

The solvents employed were:

A=0.1% v/v solution of TFA in water

B=0.1% v/v solution of TFA in MeCN.

Injection Volume: 3 mL

The UV detection was for a signal wavelength at 254 nm.

MS Conditions

MS: Waters ZQ

Ionisation mode: Alternate scan positive/negative Electrospray

Scan Range: 100 to 1000 AMU

Scan Time: 0.50 s

Inter scan Delay: 0.2 s

### Column Chromatography

Automated column chromatography was conducted on a Teledyne Isco Combiflash Rf system using RediSep Rf Silica cartridges (for normal phase), or Biotage KP-C18-HS cartridges (for reverse phase) of the correct size. Elution utilised standard HPLC grade solvents provided by Sigma Aldrich, with the desired modifier (for reverse phase) added in-house, unless otherwise stated.

### Intermediate 1

### Oxazole-5-Carboxylic Acid

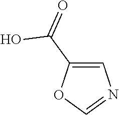

An aqueous solution of lithium hydroxide monohydrate (124.5 kg of a solution prepared from 49.44 kg lithium hydroxide monohydrate dissolved in 319 kg water, 398 mol) was added to a solution of ethyl 5-oxazolecarboxylate (54 kg, 382.7 mol) in water (54 kg) maintaining the temperature below 25° C. The reaction was stirred for 6.5 h and then conc. aqueous HCl (64.8 kg) was added maintaining the temperature below 25° C., the crystallisation cooled to 5° C. and held for 1 h. The product was filtered off, washed with cold water (88 kg), then isopropanol (171 kg) and dried under vacuum at 50° C. to give the title compound (37.88 kg, 87.5%).

¹H NMR (400 MHz, DMSO-d₆) δ ppm 13.68 (br. s., 1H), 8.59 (s, 1H), and 7.88 (s, 1H).

### Intermediate 2

### (4-Isopropylpiperazin-1-yl)(oxazol-5-yl)methanone

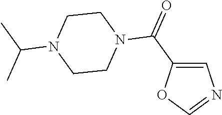 [SMILES: CC(C)N1CCN(C(=O)c2cnco2)CC1]

### Method A

Oxalyl chloride (47.7 kg, 375.8 mol) was added to a solution of oxazole-5-carboxylic acid (32.88 kg, 290.8 mol) in isopropyl acetate (144 kg) maintaining the temperature at 52-58° C. The temperature was increased to 58.5° C., stirred for 5 h and then cooled to 20° C. The reaction mixture was added to a solution of 1-(isopropyl)piperazine (41 kg, 319.8 mol) and potassium carbonate (118.4 kg) in isopropyl acetate (348 kg) and water (103 kg) maintaining the temperature below 25° C. The reaction was stirred for 15 mins, the temperature was increased to 33° C. and the organic phase washed with water (191 kg), concentrated under reduced pressure to 95 L and cooled to 20° C. n-Heptane (157 kg) was added and the crystallisation stirred for 2 h, and the product filtered off, washed with n-heptane (157 kg) and dried under vacuum 40° C. to give the title compound (57.14 kg, 88.0%).

¹H NMR (400 MHz, CDCl₃-d) δ ppm 7.94 (s, 1H), 7.56 (s, 1H), 3.91-3.73 (m, 4H), 2.75 (spt., J=6.5 Hz, 1H), 2.63-2.52 (m, 4H) and 1.06 (d, J=6.6 Hz, 6H).

### Method B

1-Isopropylpiperazine (1.06 g, 8.24 mmol) and ethyl-oxazole-5-carboxylate (1.16 g, 8.24 mmol) were added to a suspension of 5 Å molecular sieves (8.0 g) in cyclopentyl methyl ether (40 mL) and stirred for 1 h at 55° C. Lyophilised lipase TL (2.0 g) was added, the reaction mixture stirred for 28.75 h, then filtered through glass fibre paper, washed through with cyclopentyl methyl ether (3×6 mL). The combined filtrate and washings were concentrated under reduced pressure and the crude residue re-slurried in methyl cyclohexane (6 mL), filtered off, washed with methyl cyclohexane (2×5 mL), and dried under vacuum to give the title compound (1.40 g, 76%).

### Intermediate 3

### 4-Chloro-1H-indazole

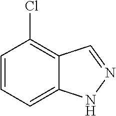

Acetic anhydride (69 kg, 675.9 mol) was added to a stirred slurry of 3-chloro-2-methylaniline (30 kg, 211.9 mol), potassium acetate (25 kg, 254.7 mol) and methyltetrahydrofuran (302 L) at 25° C. and then stirred for 2 h. Isopentyl nitrite (44.5 kg, 379.9 mol) was added to the slurry and the contents were heated to 73° C. for 18 h. The slurry was cooled to 20° C., then water (90 L) was added, and the reaction was cooled to 5° C. An aqueous solution of NaOH (105 L of a 32% w/w) was added, the solution was heated to 40° C. and stirred for 2 h. The lower aqueous phase was removed and the organic layer washed with water (150 L) and then brine (18 kg in 90 L water). The organic layer was concentrated to 90 L by atmospheric distillation and a solvent exchange to n-heptane performed by atmospheric distillation to a final volume of 240 L. The slurry was cooled to 7° C., stirred for 2 h and solid isolated by filtration. The cake was washed with heptane (2×60 L) and dried under vacuum to give the title compound (23.6 kg, 73%).

¹H NMR (400 MHz, MeOD-d₄) δ=8.08 (s, 1H), 7.48 (d, J=8.6 Hz, 1H), 7.33 (dd, J=7.5, 8.4 Hz, 1H), 7.14 (d, J=7.3 Hz, 1H)

HPLC Rₜ=1.94 min.

### Intermediate 4

### 4-Chloro-1-(tetrahydro-2H-pyran-2-yl)-1H-indazole

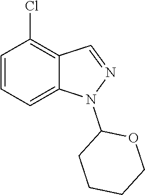 [SMILES: Clc1cccc2c1cnn2C1CCCCO1]

Trifluoroacetic acid (3.5 kg, 30.7 mol) was added to a solution of 4-chloro-1H-indazole (23 kg, 15.1 mol) and 3,4-dihydro-2H-pyran (43.1 kg, 512.7 mol) in ethyl acetate (235 L). The reaction mixture was heated to 80° C. for 4 h, then cooled to 23° C., triethylamine (3.2 kg, 31.6 mol) added and stirred for 30 min. The solvent was exchanged to isopropanol using an atmospheric distillation to a final volume of 138 L. The solution was cooled to 55° C. and water (138 L) was added maintaining the temperature. The solution was cooled to 42° C. and seeded (8 g), then cooled to 30° C., held for 10 h, and water (46 L) added, cooled to 18° C., and the slurry was stirred for 2 h then filtered off, washed with 6:1 v/v water/2-propanol (2×46 L) and dried under vacuum at 50° C. to give the title compound (28.8 kg, 80.7%).

¹H NMR (500 MHz, DMSO-d₆) δ=8.18 (s, 1H), 7.74 (d, J=8.5 Hz, 1H), 7.42 (dd, J=7.5, 8.4 Hz, 1H), 7.27 (d, J=7.3 Hz, 1H), 5.88 (dd, J=2.5, 9.5 Hz, 1H), 3.90-3.85 (m, 1H), 3.79-3.70 (m, 1H), 2.44-2.35 (m, 1H), 2.07-1.95 (m, 2H), 1.81-1.69 (m, 1H), 1.64-1.53 (m, 2H).

### Intermediate 5

### (4-Isopropylpiperazin-1-yl)(2-(1-(tetrahydro-2H-pyran-2-yl)-1H-indazol-4-yl)oxazol-5-yl)methanone

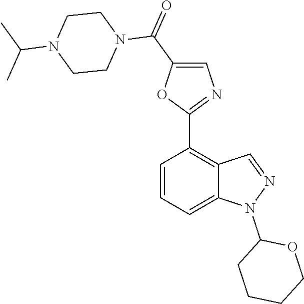 [SMILES: CC(C)N1CCN(C(=O)c2cnc(-c3cccc4c3cnn4C3CCCCO3)o2)CC1]

### Method A

Palladium chloride (0.74 kg, 4.2 mol) and XPhos (4.36 kg, 9.1 mol) was suspended in cyclopentyl methyl ether (372 L). 4-Chloro-1-(tetrahydro-2H-pyran-2-yl)-1H-indazole (36 kg, 152.1 mol), (4-Isopropylpiperazin-1-yl)(oxazol-5-yl)methanone (34.78 kg, 155.8 mol) and potassium carbonate (325 mesh, 35.64 kg, 257.9 mol) were added and rinsed in with cyclopentyl methyl ether (2.58 kg). A solution of pivalic acid (9.294 kg, 91.0 mol) dissolved in cyclopentyl methyl ether (10 L) was added followed by a rinse of cyclopentyl methyl ether (10 L). The reaction was vacuum degassed and back-filled with nitrogen three times, then heated to reflux for 5 h and the contents cooled to 40° C. The reaction mixture was washed with water (144 L) then 5% w/v aqueous sodium chloride solution (151.2 kg) and the organic phase was concentrated at atmospheric pressure to 288 L. The reaction mixture was filtered into another vessel and the filter washed with cyclopentyl methyl ether (36 L), then distilled to 108 L under atmospheric pressure. Methyl cyclohexane (162 L) was added to the vessel maintaining the temperature at 75° C., the contents were then cooled to 62-65° C. and the crystallisation seeded with (4-isopropylpiperazin-1-yl)(2-(1-(tetrahydro-2H-pyran-2-yl)-1H-indazol-4-yl)oxazol-5-yl)methanone (90 g) slurried in chilled methyl cyclohexane (0.52 L). The crystallisation was held at 62° C. for 30 mins, cooled to 7° C. and then held at 7° C. overnight. The product was filtered off, washed with methyl cyclohexane (2×72 L) and dried in a vacuum oven at 50° C. to give the title compound (57.2 kg, 88.9%).

¹H NMR (400 MHz, DMSO-d₆) δ ppm 8.59 (s, 1H), 8.00 (d, J=8.6 Hz, 1H), 7.95 (s, 1H), 7.91 (d, J=7.3 Hz, 1H), 7.62 (dd, J=7.3, 8.3 Hz, 1H), 5.97 (dd, J=2.0, 9.5 Hz, 1H), 4.03-3.85 (m, 1H), 3.84-3.70 (m, 1H), 3.66 (br. s., 4H), 2.72 (spt., J=6.5 Hz, 1H), 2.57-2.40 (m, 5H), 2.11-1.96 (m, 2H), 1.85-1.68 (m, 1H), 1.68-1.47 (m, 2H), 0.99 (d, J=6.4 Hz, 6H)

### Method B

4-Chloro-1-(tetrahydro-2H-pyran-2-yl)-1H-indazole (10 g, 42.2 mmol), (4-Isopropylpiperazin-1-yl)(oxazol-5-yl)methanone (9.67 g, 43.3 mmol), potassium carbonate (325 mesh, 9.93 g, 71.8 mmol) and pivalic acid (2.59 g, 25.3 mmol) were suspended in CPME (90 mL). The reaction was stirred for 10 mins at room temperature and then vacuum degassed and back-filled with nitrogen three times. Palladium chloride (206 mg, 1.16 mmol) and XPhos (1.21 g, 2.53 mmol) were added and rinsed in with CPME (10 mL). The reaction mixture was vacuum degassed and back-filled with nitrogen three times, then heated to reflux for 5 h and the contents cooled back to 50° C. The reaction mixture was washed with 3% w/w aqueous sodium chloride solution (30 mL), then 20% w/w aqueous sodium chloride solution (30 mL) and the organic phase was concentrated at atmospheric pressure down to 80 mL. The reaction mixture was cooled to room temperature and held overnight. The reaction mixture was then filtered into another vessel and the filter washed with CPME (20 mL), then distilled down to 30 mL under atmospheric pressure. The contents were cooled to 80° C. and methyl cyclohexane (45 mL) was added to the vessel maintaining the temperature at 75° C., the contents were then cooled to 62-65° C. and the crystallisation seeded with (4-isopropylpiperazin-1-yl)(2-(1-(tetrahydro-2H-pyran-2-yl)-1H-indazol-4-yl)oxazol-5-yl)methanone. The crystallisation was held at 63° C. for 30 mins, cooled to 5° C. over 6 h and then held at 5° C. overnight. The product was filtered off, washed with chilled methyl cyclohexane (2×20 mL) and dried in a vacuum oven at 50° C. to give the title compound (13.52 g, 76%).

¹H NMR (400 MHz, DMSO-d₆) δ ppm 8.59 (s, 1H), 8.00 (d, J=8.6 Hz, 1H), 7.95 (s, 1H), 7.91 (d, J=7.3 Hz, 1H), 7.62 (dd, J=7.3, 8.3 Hz, 1H), 5.97 (dd, J=2.0, 9.5 Hz, 1H), 4.03-3.85 (m, 1H), 3.84-3.70 (m, 1H), 3.66 (br. s., 4H), 2.72 (spt., J=6.5 Hz, 1H), 2.57-2.40 (m, 5H), 2.11-1.96 (m, 2H), 1.85-1.68 (m, 1H), 1.68-1.47 (m, 2H), 0.99 (d, J=6.4 Hz, 6H)

### Intermediate 6

### (4-Isopropylpiperazin-1-yl)(2-(1-(tetrahydro-2H-pyran-2-yl)-6-(4,4,5,5-tetramethyl-1,3,2-dioxaborolan-2-yl)-1H-indazol-4-yl)oxazol-5-yl)methanone

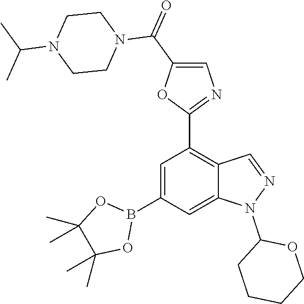 [SMILES: CC(C)N1CCN(C(=O)c2cnc(-c3cc(B4OC(C)(C)C(C)(C)O4)cc4c3cnn4C3CCCCO3)o2)CC1]

Pinacolborane (40.80 kg, 318.8 mol) was added to a stirred solution of (4-isopropylpiperazin-1-yl)(2-(1-(tetrahydro-2H-pyran-2-yl)-1H-indazol-4-yl)oxazol-5-yl)methanone (54.00 kg, 127.5 mol), (1,5-cyclooctadiene)(methoxy)iridium(I) dimer (0.864 kg, 1.30 mol) and 3,4,7,8-tetramethyl-1,10-phenanthroline (1.51 kg, 6.39 mol) in THF (243.2 kg) at 20° C. The reaction was heated to reflux for 8 h, then cooled to 20° C. and transferred into a vessel containing isopropanol (253.8 kg), washed through with THF (23.8 kg). The solvent was distilled to 270 L at 200 mbar and isopropanol (253.8 kg) was added and then redistilled to 324 L at 100 mbar. The crystallisation is heated to 40° C. for 4 h, cooled to 20° C., stirred overnight, filtered off, washed with isopropanol (84.8 kg), and dried in a vacuum oven at 50° C. to give the title compound (57.35 kg, 82.8%).

¹H NMR (400 MHz, CDCl₃-d) δ ppm 8.70 (s, 1H), 8.40 (s, 1H), 8.17 (s, 1H), 7.74 (s, 1H), 5.85 (dd, J=2.4, 9.5 Hz, 1H), 4.11-4.03 (m, 1H), 3.97-3.77 (m, 5H), 2.78 (spt., J=6.4 Hz, 1H), 2.70-2.59 (m, 5H), 2.24-2.14 (m, 1H), 2.14-2.03 (m, 1H), 1.86-1.72 (m, 2H), 1.72-1.62 (m, 1H), 1.40 (s, 12H), 1.08 (d, J=6.4 Hz, 6H).

### Intermediate 7

### Methyl 5-(4-(5-(4-isopropylpiperazine-1-carbonyl)oxazol-2-yl)-1-(tetrahydro-2H-pyran-2-yl)-1H-indazol-6-yl)-2-methoxynicotinate

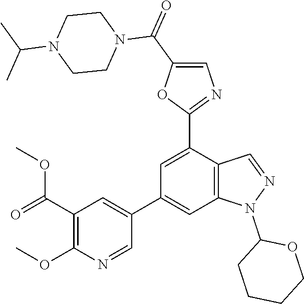 [SMILES: COC(=O)c1cc(-c2cc(-c3ncc(C(=O)N4CCN(C(C)C)CC4)o3)c3cnn(C4CCCCO4)c3c2)cnc1OC]

A flask was charged with (4-isopropylpiperazin-1-yl)(2-(1-(tetrahydro-2H-pyran-2-yl)-6-(4,4,5,5-tetramethyl-1,3,2-dioxaborolan-2-yl)-1H-indazol-4-yl)oxazol-5-yl)methanone (4.91 g, 8.94 mmol), methyl 5-bromo-2-methoxynicotinate (2.00 g, 8.13 mmol), PdCl₂(dppf) (0.595 g, 0.813 mmol), and Na₂CO₃ (2.58 g, 24.4 mmol). 1,4-Dioxane (100 mL) and water (25 mL) were then added and the reaction stirred. The vessel was degassed and purged with nitrogen three times and then heated at 80° C. for 30 min. The reaction was cooled to room temperature, filtered through celite and concentrated in vacuo. The residue was taken up in EtOAc (100 mL) and water (20 mL). The layers were separated and the organics washed with water (3×20 mL) and brine (3×20 mL). The organics were dried through a hydrophobic frit and concentrated in vacuo. The residue was purified by automated column chromatography on silica gel (0-30% EtOH:EtOAc). Desired fractions were combined and the solvent removed in vacuo to give the title product product as a brown foam (4.5 g).

LCMS (Method A): Rₜ=0.77 min, [M+H⁺] 589.6.

### Intermediate 8

### Methyl 5-(4-(5-((4-isopropylpiperazin-1-yl)methyl)oxazol-2-yl)-1-(tetrahydro-2H-pyran-2-yl)-1H-indazol-6-yl)-2-methoxynicotinate

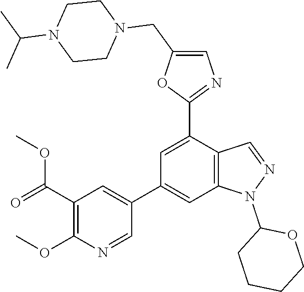 [SMILES: COC(=O)c1cc(-c2cc(-c3ncc(CN4CCN(C(C)C)CC4)o3)c3cnn(C4CCCCO4)c3c2)cnc1OC]

To a flask containing methyl 5-(4-(5-(4-isopropylpiperazine-1-carbonyl)oxazol-2-yl)-1-(tetrahydro-2H-pyran-2-yl)-1H-indazol-6-yl)-2-methoxynicotinate (100 mg, 0.170 mmol) and tris(triphenylphosphine)rhodium(I) carbonyl hydride (1.8 mg, 2.0 μmol) was added THF (0.2 mL) and diphenylsilane (0.073 mL, 0.39 mmol). Effervescence was observed upon addition of the silane. The reaction was left to stir at room temperature. At 17 h, another portion of catalyst (2.0 mg, 2.2 μmol) and silane (0.075 mL, 0.40 mmol) were added. After a further 1.5 h, the reaction was diluted with 2 mL ether and the product extracted to aqueous HCl (1 M, 6×1 mL washings). The organics were discarded and the aqueous basified to pH 8 by the addition of sodium bicarbonate, and the product extracted to EtOAc (2×25 mL). The organics were dried through a hydrophobic frit, concentrated in vacuo and the residue purified by automated column chromatography on silica gel (0-20% MeOH:DCM). Desired fractions were combined and the solvent removed in vacuo to give the title product as a brown gum (62 mg).

LCMS (Method A): Rₜ=0.78 min, [M+H⁺] 575.6.

### Example 1

### Methyl 5-(4-(5-((4-isopropylpiperazin-1-yl)methyl)oxazol-2-yl)-1H-indazol-6-yl)-2-methoxynicotinate, formic acid salt

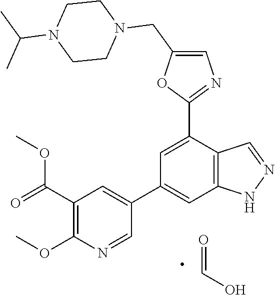

To a flask containing methyl 5-(4-(5-((4-isopropylpiperazin-1-yl)methyl)oxazol-2-yl)-1-(tetrahydro-2H-pyran-2-yl)-1H-indazol-6-yl)-2-methoxynicotinate (55 mg, 0.096 mmol) in anhydrous methanol (2 mL) under nitrogen was added chlorotrimethylsilane (TMSCl) (0.15 mL, 1.2 mmol). The reaction was stirred at 40° C. for 2 h. Triethylamine (0.164 mL, 1.17 mmol) was added to the vessel and the reaction cooled to room temperature. The solvent was removed in vacuo and the residue purified by automated column chromatography on silica gel, pre-treated with triethylamine (0-15% MeOH:DCM). The product was further purified by MDAP (Method A). Desired fractions were combined and the solvent removed under a stream of nitrogen to afford the the title product as a white gum (42 mg). Formic acid salt seen from communal drying apparatus.

LCMS (Method A): Rₜ=0.60 min, [M+H⁺] 491.5.

### Example 2

### 5-(4-(5-((4-Isopropylpiperazin-1-yl)methyl)oxazol-2-yl)-1H-indazol-6-yl)-2-methoxynicotinic acid

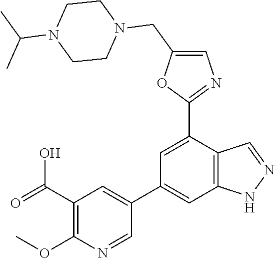 [SMILES: COc1ncc(-c2cc(-c3ncc(CN4CCN(C(C)C)CC4)o3)c3cn[nH]c3c2)cc1C(=O)O]

To a flask containing methyl 5-(4-(5-((4-isopropylpiperazin-1-yl)methyl)oxazol-2-yl)-1-(tetrahydro-2H-pyran-2-yl)-1H-indazol-6-yl)-2-methoxynicotinate (200 mg, 0.350 mmol) in anhydrous methanol (8 mL) under nitrogen was added TMSCl (0.45 mL, 3.5 mmol). The reaction was stirred at 40° C. for 16 h. Aqueous NaOH (2 M, 1.74 mL, 3.48 mmol) was then added slowly to neutralise the reaction, and the pH adjusted to pH 11 by the addition of more aqueous NaOH (2 M). The reaction was heated at 65° C. for 1 h, then cooled to room temperature and concentrated in vacuo. The residue was taken up in approximately 1:1 DMSO/water adjusted to pH 10 with ammonium bicarbonate and purified by automated reverse phase column chromatography on C18 silica gel (5-95% acetonitrile:water adjusted to pH 10 with ammonium bicarbonate). Desired fractions were collected and the solvent removed in vacuo to afford the title product as a beige solid (153 mg).

LCMS (Method A): Rₜ=0.54 min, [M+H⁺] 477.5.

### Example 3

### 2-Nitrophenyl 5-(4-(5-((4-isopropylpiperazin-1-yl)methyl)oxazol-2-yl)-1H-indazol-6-yl)-2-methoxynicotinate, formic acid salt

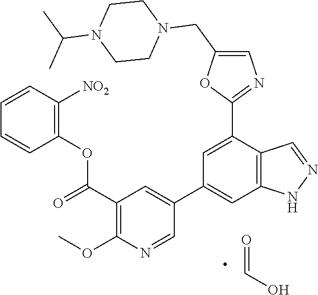

HATU (96 mg, 0.252 mmol), 5-(4-(5-((4-isopropylpiperazin-1-yl)methyl)oxazol-2-yl)-1H-indazol-6-yl)-2-methoxynicotinic acid (100 mg, 0.210 mmol), and DIPEA (0.073 mL, 0.42 mmol) were stirred in DMF (1.5 mL) at room temperature for 10 min prior to the addition of o-nitrophenol (44 mg, 0.32 mmol). The reaction was left to stir at room temperature. After 1 h, the reaction was diluted to 2 mL with DMSO and purified by MDAP (Method B). Desired fractions were combined, concentrated in vacuo and then dried fully using a Vapourtec V10 system to afford the product as an off-white solid (34 mg).

LCMS (Method A): Rₜ=0.74 min, [M+H⁺] 598.2.

### Example 4

### 4-Nitrophenyl 5-(4-(5-((4-isopropylpiperazin-1-yl)methyl)oxazol-2-yl)-1H-indazol-6-yl)-2-methoxynicotinate

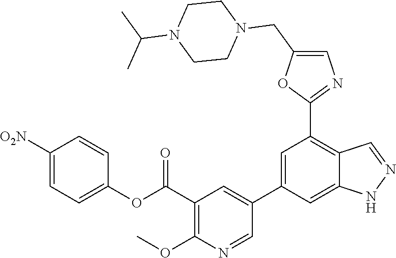 [SMILES: COc1ncc(-c2cc(-c3ncc(CN4CCN(C(C)C)CC4)o3)c3cn[nH]c3c2)cc1C(=O)Oc1ccc([N+](=O)[O-])cc1]

HATU (96 mg, 0.25 mmol), 5-(4-(5-((4-isopropylpiperazin-1-yl)methyl)oxazol-2-yl)-1H-indazol-6-yl)-2-methoxynicotinic acid (100 mg, 0.210 mmol) and DIPEA (0.073 mL, 0.42 mmol) were stirred in DMF (1.5 mL) at room temperature for 10 min prior to the addition of 4-nitrophenol (44 mg, 0.32 mmol). The reaction was then left to stir at room temperature. After 1 h, the reaction was diluted to 2 mL with DMSO and purified by MDAP (Method B). Desired fractions were combined, concentrated in vacuo and then dried fully using the Vapourtec V10 to afford a yellow solid. The sample was taken up in DMSO (1 mL) and purified further by MDAP (Method D). Desired fractions were dried under flowing nitrogen at 40° C. overnight to afford the product as a TFA salt. The sample was partitioned between DCM (2 mL) and distilled water (2 mL). Saturated aqueous NaHCO₃ was added dropwise to adjust the pH to 10, and the product was extracted to the organic layer. The aqueous was extracted further with 3×2 mL DCM. The organics were then dried through a hydrophobic frit, concentrated by nitrogen blowdown and dried further in a vacuum oven at 40° C. for 2 h to afford the title product as a yellow solid (17 mg).

LCMS (Method A): Rₜ=0.76 min, [M+H⁺] 598.2.

### Example 5

### 2-(Trifluoromethyl)phenyl 5-(4-(5-((4-isopropylpiperazin-1-yl)methyl) oxazol-2-yl)-1H-indazol-6-yl)-2-methoxynicotinate

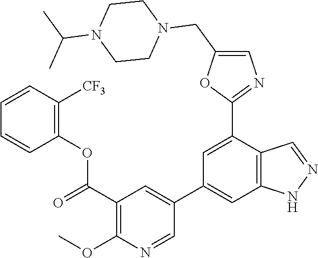 [SMILES: COc1ncc(-c2cc(-c3ncc(CN4CCN(C(C)C)CC4)o3)c3cn[nH]c3c2)cc1C(=O)Oc1ccccc1C(F)(F)F]

5-(4-(5-((4-Isopropylpiperazin-1-yl)methyl)oxazol-2-yl)-1H-indazol-6-yl)-2-methoxynicotinic acid (100 mg, 0.210 mmol), o-(trifluoromethyl)phenol (51 mg, 0.32 mmol) and (PyBOP) (120 mg, 0.231 mmol) and were stirred in DMF (1.5 mL) at room temperature. DIPEA (0.073 mL, 0.42 mmol) was then added dropwise to the stirring reaction and the reaction left to stir at room temperature. After 5 minutes, the reaction was diluted to 3 mL with DMSO, and purified directly, without workup by MDAP (Method A, three consecutive purifications required). Desired fractions were combined, and the volatile solvents removed in vacuo. The pH of the remaining aqueous was adjusted to >10 with aq. NH₄OH, and the product extracted to DCM (5×10 mL). The organics were dried through a hydrophobic frit and concentrated in vacuo to afford the title product as an off-white solid (16 mg).

LCMS (Method A): Rₜ=0.80 min, [M+H⁺] 621.2.

### Example 6

### 4-(Trifluoromethyl)phenyl 5-(4-(5-((4-isopropylpiperazin-1-yl)methyl)oxazol-2-yl)-1H-indazol-6-yl)-2-methoxynicotinate, formic acid salt

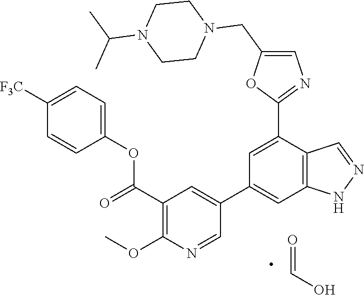

p-(Trifluoromethyl)phenol (51 mg, 0.32 mmol), PyBOP (120 mg, 0.231 mmol) and 5-(4-(5-((4-isopropylpiperazin-1-yl)methyl)oxazol-2-yl)-1H-indazol-6-yl)-2-methoxynicotinic acid (100 mg, 0.210 mmol) were stirred in DMF (1.5 mL) at room temperature. DIPEA (0.073 mL, 0.42 mmol) was then added dropwise to the stirring reaction and the reaction left to stir at room temperature. After 3 h, the reaction was diluted to 3 mL and the product purified directly, without workup by MDAP (Method C). Desired fractions were combined and the solvent removed by nitrogen blowdown at 40° C. The solid was taken up in DMSO (1 mL) and further purified by MDAP (Method B). Desired fractions were combined, and the solvent removed by nitrogen blowdown to afford the title product as a white solid (55 mg).

LCMS (Method A): Rₜ=0.85 min, [M+H⁺] 621.2.

### Example 7

### 4-Fluorophenyl 5-(4-(5-((4-isopropylpiperazin-1-yl)methyl)oxazol-2-yl)-1H-indazol-6-yl)-2-methoxynicotinate

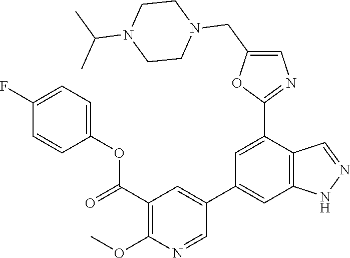 [SMILES: COc1ncc(-c2cc(-c3ncc(CN4CCN(C(C)C)CC4)o3)c3cn[nH]c3c2)cc1C(=O)Oc1ccc(F)cc1]

5-(4-(5-((4-Isopropylpiperazin-1-yl)methyl)oxazol-2-yl)-1H-indazol-6-yl)-2-methoxynicotinic acid (100 mg, 0.210 mmol), p-fluorophenol (35 mg, 0.32 mmol) and PyBOP (120 mg, 0.231 mmol) and were stirred in DMF (1.5 mL) at room temperature. DIPEA (0.073 mL, 0.42 mmol) was then added dropwise to the stirring reaction and the reaction left to stir at room temperature. After 10 minutes, the reaction was diluted to 3 mL with DMSO, and purified directly, without workup by MDAP (Method C). Desired fractions were combined, and the volatile solvents removed in vacuo. The pH of the remaining aqueous was adjusted to >10 with aq. NH₄OH, and the product extracted to DCM (5×10 mL). The organics were dried through a hydrophobic frit, concentrated in vacuo and dried in a vacuum oven at 40° C. for 1 h to afford the title product as an off-white solid (55 mg).

LCMS (Method A): Rₜ=0.77 min, [M+H⁺] 571.1.

### Example 8

### Phenyl 5-(4-(5-((4-isopropylpiperazin-1-yl)methyl)oxazol-2-yl)-1H-indazol-6-yl)-2-methoxynicotinate

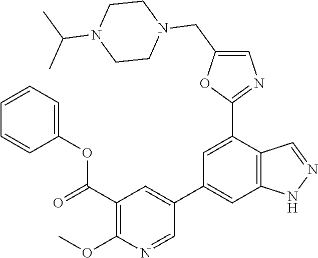 [SMILES: COc1ncc(-c2cc(-c3ncc(CN4CCN(C(C)C)CC4)o3)c3cn[nH]c3c2)cc1C(=O)Oc1ccccc1]

5-(4-(5-((4-Isopropylpiperazin-1-yl)methyl)oxazol-2-yl)-1H-indazol-6-yl)-2-methoxynicotinic acid (100 mg, 0.210 mmol), phenol (30 mg, 0.32 mmol) and PyBOP (120 mg, 0.231 mmol) and were stirred in DMF (1.5 mL) at room temperature. DIPEA (0.073 mL, 0.42 mmol) was then added dropwise to the stirring reaction and the reaction left to stir at room temperature. After 5 min, the reaction was diluted to 3 mL with DMSO, and purified directly, without workup by MDAP (Method C). Desired fractions were combined, and the volatile solvents removed in vacuo. The pH of the remaining aqueous was adjusted to >10 with aq. NH₄OH, and the product extracted to DCM (5×10 mL). The organics were dried through a hydrophobic frit, concentrated in vacuo to afford the title product as an off-white solid (29 mg).

LCMS (Method A): Rₜ=0.73 min, [M+H⁺] 553.2.

### Example 9

### 2,4-Dimethylphenyl 5-(4-(5-((4-isopropylpiperazin-1-yl)methyl)oxazol-2-yl)-1H-indazol-6-yl)-2-methoxynicotinate

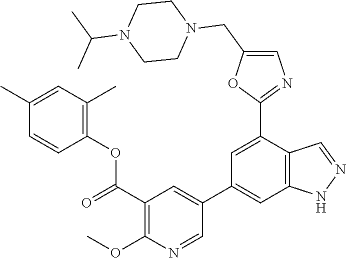 [SMILES: COc1ncc(-c2cc(-c3ncc(CN4CCN(C(C)C)CC4)o3)c3cn[nH]c3c2)cc1C(=O)Oc1ccc(C)cc1C]

5-(4-(5-((4-Isopropylpiperazin-1-yl)methyl)oxazol-2-yl)-1H-indazol-6-yl)-2-methoxynicotinic acid (100 mg, 0.210 mmol), 2,4-dimethylphenol (0.039 mL, 0.32 mmol) and PyBOP (120 mg, 0.231 mmol) and were stirred in DMF (1.5 mL) at room temperature. DIPEA (0.073 mL, 0.42 mmol) was then added dropwise to the stirring reaction and the reaction left to stir at room temperature. After 2 h, the reaction was diluted to 3 mL, and purified directly, without workup, by MDAP (Method C). Desired fractions were combined, and the volatile solvents removed in vacuo. The pH of the remaining aqueous was adjusted to >10 with aq. NH₄OH, and the product extracted to DCM (5×10 mL). The organics were dried through a hydrophobic frit, concentrated in vacuo and dried in a vacuum oven at 40° C. overnight to afford the title product as an off-white solid (14 mg).

LCMS (Method A): Rₜ=0.82 min, [M+H⁺] 581.2.

### Example 10

### 4-Methoxyphenyl 5-(4-(5-((4-isopropylpiperazin-1-yl)methyl)oxazol-2-yl)-1H-indazol-6-yl)-2-methoxynicotinate

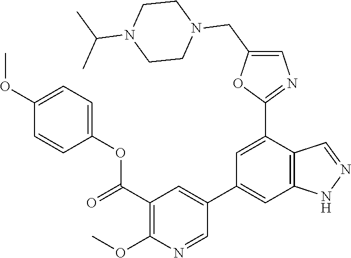 [SMILES: COc1ccc(OC(=O)c2cc(-c3cc(-c4ncc(CN5CCN(C(C)C)CC5)o4)c4cn[nH]c4c3)cnc2OC)cc1]

5-(4-(5-((4-Isopropylpiperazin-1-yl)methyl)oxazol-2-yl)-1H-indazol-6-yl)-2-methoxynicotinic acid (100 mg, 0.210 mmol), p-methoxyphenol (0.039 mg, 0.31 mmol) and PyBOP (120 mg, 0.231 mmol) were stirred in DMF (1.5 mL) at room temperature. DIPEA (0.073 mL, 0.42 mmol) was then added dropwise to the stirring reaction and the reaction left to stir at room temperature. After 1 h, the reaction was diluted to 3 mL with DMSO, and purified directly, without workup, by MDAP (Method C). Desired fractions were combined, and the volatile solvents removed in vacuo. The pH of the remaining aqueous was adjusted to >10 with aq. NH₄OH, and the product extracted to DCM (5×10 mL). The organics were dried through a hydrophobic frit, concentrated in vacuo and dried in a vacuum oven at 40° C. overnight to afford the title product as an off-white solid (22 mg).

LCMS (Method A): Rₜ=0.73 min, [M+H⁺] 583.6.

### Example 11

### 2,2,2-Trifluoroethyl 5-(4-(5-((4-isopropylpiperazin-1-yl)methyl)oxazol-2-yl)-1H-indazol-6-yl)-2-methoxynicotinate

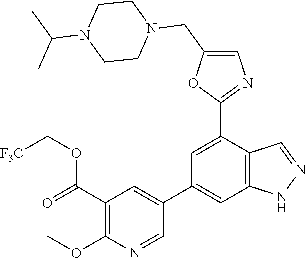 [SMILES: COc1ncc(-c2cc(-c3ncc(CN4CCN(C(C)C)CC4)o3)c3cn[nH]c3c2)cc1C(=O)OCC(F)(F)F]

5-(4-(5-((4-Isopropylpiperazin-1-yl)methyl)oxazol-2-yl)-1H-indazol-6-yl)-2-methoxynicotinic acid (100 mg, 0.210 mmol), 2,2,2-trifluoroethanol (0.023 mL, 0.32 mmol) and PyBOP (120 mg, 0.231 mmol) were stirred in DMF (1.5 mL) at room temperature. DIPEA (0.073 mL, 0.42 mmol) was then added dropwise to the stirring reaction and the reaction left to stir at room temperature. After 30 min, the reaction was diluted to 3 mL with DMSO and purified by MDAP (Method C). Desired fractions were combined, and the volatile solvents removed in vacuo. The pH of the remaining aqueous was adjusted to >10 by the addition of ammonium hydroxide, and the product extracted to DCM (5×10 mL). The organics were dried through a hydrophobic frit and concentrated in vacuo to afford the title product as an off-white solid (22 mg).

LCMS (Method A): Rₜ=0.70 mins, [M+H⁺]=559.6

### PI3K HTRF Assay

The binding of compounds to PI3K-alpha/beta/delta/gamma is determined by homogeneous time resolved fluorescence (HTRF) assays as follows;

Briefly, solid compound is dissolved in 100% DMSO at a concentration of 2 mM. Dilutions are prepared in 100% DMSO using a 1 in 4 serial step dilution. The dilutions are transferred to black low volume Greiner assay plates ensuring that the DMSO concentration is constant across the plate at 1% (0.1 μL/well).

PI3K Reaction Buffer (contains 50 mM HEPES pH 7.0 (NaOH), 150 mM NaCl, 10 mM MgCl₂, 2.3 mM sodium cholate, 10 pM CHAPS made up in milliQ water). Fresh DTT is added at a final concentration of 1 mM on the day of use. Wortmannin at a concentration sufficient to produce 100% inhibition (8.33 pM) is added to column 18 of compound plates.

Enzyme solutions: 1× PI3K Assay Buffer containing:
550 pM PI3K-Alpha enzyme (275 pM final assay concentration)
800 pM PI3K-Beta enzyme (400 pM final assay concentration)
3 nM PI3K-Delta enzyme (1.5 nM final assay concentration)
10 nM PI3K-Gamma enzyme (5 nM final assay concentration)

These concentrations are optimal to achieve a signal:background of between 1.5-4.5. The enzyme solution is added to columns 1-24 (3 μL/well) and plates are incubated for 15 minutes at room temperature.

Substrate solution: 1× PI3K Assay Buffer containing:
PI3K-Alpha: 500 μM ATP, 20 μM PIP2 and 120 nM biotin-PIP3. (Final assay concentrations are 250 μM ATP, 10 μM PIP2 (both at Kₘ) and 40 nM biotin-PIP3)
PI3K-Beta: 800 μM ATP, 20 μM PIP2 and 120 nM biotin-PIP3. (Final assay concentrations are 400 μM ATP, 10 μM PIP2 (both at Kₘ) and 40 nM biotin-PIP3)
PI3K-Delta: 160 μM ATP, 20 μM PIP2 and 120 nM biotin-PIP3. (Final assay concentrations are 80 μM ATP, 10 μM PIP2 (both at Kₘ) and 40 nM biotin-PIP3)
PI3K-Gamma: 30 μM ATP, 20 μM PIP2 and 120 nM biotin-PIP3. (Final assay concentrations are 15 μM ATP, 10 μM PIP2 (both at Kₘ) and 40 nM biotin-PIP3)

This is added to all wells and plates are incubated for 1 hour at room temperature.

Detection solution: PI3K Detection Buffer (contains 50 mM HEPES pH 7.0 (HCl), 150 mM NaCl, 2.3 mM sodium cholate, 10 μM CHAPS, 240 mM potassium fluoride) containing 2 mM DTT (2× final assay concentration), 90 nM GRP-1 PH domain, 300 nM Streptavidin-APC and 24 nM Europium-anti-GST (6× final assay concentrations).

This is mixed left at room temperature (protected from light).

STOP solution: PI3K STOP Buffer (contains 50 mM HEPES pH 7.0 (HCl), 150 mM NaCl, 2.3 mM sodium cholate, 10 μM CHAPS, 150 mM EDTA).

Detection solution is diluted 1:1 with STOP solution and added to all wells (3 μL/well). Plates are covered and incubated on the bench for 45-60 minutes.

Plates are read on a PerkinElmer Envision, measuring TR-FRET between the complex formed between the GST-tagged PH domain and biotinylated PIP3 which both recruit fluorophores (Europium-labelled anti-GST & Strep-APC respectively). In the presence of an inhibitor, this complex is disrupted by the competitive action of non-biotinylated PIP3 (formed in the assay by the phosphorylation of PIP2 by the kinase & ATP). From this, the ratio of acceptor/donor was calculated (λₑₓ=317 nm, Δₑₘ donor=615 nm, Δₑₘ acceptor=665 nm) and used for data analysis.

pIC₅₀

| Example | PI3Kδ | PI3Kα | PI3Kβ | PI3Kγ |
|---|---|---|---|---|
| 1 | 7.4 | 5 | <4.5 | <4.5 |
| 2 | 7.9 | 4.9 | 4.7 | 4.8 |
| 3^{a} | 8.3 | 7.1 | 6.7 | 5.5 |
| 4^{a} | 9.2 | 8.2 | 7.2 | 5.8 |
| 5 | 8.2 | 6 | 5.7 | 5.1 |
| 6 | 8.4 | 5.7 | 5.2 | 4.6 |
| 7* | 8.2 | 5.5 | 5.3 | 4.8 |
| 8 | 8.2 | 5.6 | 5.4 | 4.9 |
| 9 | 6.4 | 5.1 | 4.8 | 5 |
| 10 | 7.3 | 4.8 | 4.9 | 5 |
| 11 | 6.5 | 4.8 | <4.5 | 4.7 |
| Wortmannin | 8.3 | 8.1 | 8.0 | 8.2 |

*= Median values reported
^{a}= Data reported from N = 1 due to instability

### Protein Mass Spectrometry

### Covalent Adduct Assessment

Compounds were incubated with 1 μM protein in a 10:1 inhibitor:protein ratio in buffer containing 50 mM HEPES, pH 7.0 (NaOH), 150 mM NaCl, 10 mM MgCl₂, 2.3 mM cholate, 10 μM CHAPS. After the desired time, the assays were quenched with a 10% formic acid solution, and analysed by LCMS using an Agilent 6224 TOF LC/MS equipped with Agilent 1200 Series autosampler. Analytes were separated on a Polymer Labs (Agilent) PLRP-S column (50 mm×1 mm internal diameter, 5 μm particle size). LC conditions were 0.5 mL·min⁻¹ flow rate, 70° C., 10 μL injection volume. Gradient elution with (A) water containing 0.2% (v/v) formic acid and (B) acetonitrile containing 0.2% (v/v) formic acid. Gradient conditions were initially 10% B, increasing to 30% after 0.5 min, and then linearly to 80% B over 4.5 min, prior to a 1.2 min flush with 100% B and then a 1.9 minute equilibration with 10% B prior to the next injection. Mass spectra were then deconvoluted using Agilent MassHunter Qualitative Analysis version B.06.00 over the mass range 70-170 kDa to obtain the intact protein mass.

### Competition Experiment

A potent reversible ATP competitive inhibitor was incubated with 1 μM protein in a 10:1 inhibitor:protein ratio in buffer containing 50 mM HEPES, pH 7.0 (NaOH), 150 mM NaCl, 10 mM MgCl₂, 2.3 mM cholate, 10 μM CHAPS at room temperature. After 15 min, Example 7 was added in a 2:1 inhibitor:protein ratio, and the samples incubated at room temperature. The assay was quenched at 5 min and analysed using the procedure detailed above.

### Mass Spectrometry Data

The above assays showed that Example 7 covalently modified PI3Kδ when incubated overnight in a 10-fold excess of inhibitor to protein. Furthermore, no additional adducts were observed after this incubation period, indicating that the reaction occured at a specific site. This modification was also observed after only 5 minutes in a second assay with only 2:1 inhibitor:protein. Preincubation of the enzyme with a potent ATP competitive ligand (10:1 inhibitor:enzyme) for 15 minutes, prior to the addition of 2 equivalents (relative to enzyme) of the covalent inhibitor prevented covalent modification of the protein, indicating that covalent adduct formation occured in the ATP binding pocket of PI3Kδ. A control assay with reversibly binding acid, Example 2 showed no modification.

### PI31δ Jump Dilution Experiment

Irreversibility of the PI3Kδ-Inhibitor complex, and reversibility of the non-covalent controls, was determined by jump-dilution assay as follows:

Briefly, solid compound was dissolved in 100% DMSO at a concentration of 5 mM. This was then dispensed into wells of a black low volume Greiner assay plate to ensure an incubation concentration equal to 10×IC₅₀, or the concentration of 100×PI3Kδ final enzyme final assay concentration—Example 7: 1 μM, Example 1: 0.6 μM, Wortmannin: 0.6 μM and Example 2: 0.6 μM. Assays were carried out in triplicate.

PI3Kδ enzyme was made up to a 100× assay concentration (final assay concentration=6 nM) in buffer solution (contains 50 mM HEPES pH 7.0 (NaOH), 150 mM NaCl, 10 mM MgCl₂, 2.3 mM sodium cholate, 10 μM CHAPS made up in milliQ water).

100× enzyme solution was added to compound wells (20 μL/well), and incubated for 15 mins, after which point the assay was diluted 100-fold with commercially available ADP QUEST substrate solution, available from DiscoverX (calatog number: 90-0071), (3200 μL DiscoverX Reagent A, 6400 μL DiscoverX Reagent B, 1 mM ATP and 40 μM PIP2). Production of fluorescence signal was then measured immediately using a Tecan Safire II plate reader at 30 second intervals for 30 minutes (λₑₓ=544 nm, λₑₘ=590 nm). These data were then analysed graphically to determine % recovery of activity, relative to high (no inhibitor) and low (no protein) controls.

### Jump Dilution Data

The above experiment confirmed the irreversibility of Example 7 at PI3Kδ. The progress curve for this compound followed, very closely, that of the known irreversible PI3K inhibitor, wortmannin, with no recovery of activity observed after 30 minutes. Reversible controls, Examples 1 and 2, showed the expected percentage recovery, based on their measured IC₅₀ values at PI3Kδ, and the concentration required to achieve an incubation concentration equal to or greater than the concentration of enzyme.

### Cellular Washout

### Method

CD4+T cells were isolated from leukodepletion filter samples taken from healthy volunteers using Histopaque 1077 (Sigma Aldrich). Briefly, blood was allowed to drip into a 50 mL falcon tube and mixed with PBS (40 mL). 20 mL of diluted blood was then slowly layered onto 15 mL of Histopaque in a 50 mL falcon tube, which was then centrifuged at 800 rcf for 20 min at room temperature. The PBMC layer was then collected with a pasteur pipette into a new 50 mL falcon tube, and up to 50 mL of PBS was added. Cells were centrifuged at 300-400 rcf for 10 min at room temperature, supernatant was discarded and a second washing step was performed with PBS. Cells were then resuspended in 40 mL PBS+0.5% Bovine Serum Albumin (BSA). Isolation of CD4+T cells was performed according to manufacturer's protocol (Miltenyi Biotec, 130-096-533). 1×10⁶ CD4+T cells were dispensed into a 24 well plate with a final volume of 1 mL per well. Compounds at 1000× the final assay concentration, or DMSO, were then added to wells and cells were incubated for 2 h. 2×10⁵ cells and media was added to a 96-well plate (in duplicate) and classified as no-wash. For the washout, the remaining cells and supernatant were transferred to a 1 mL eppendorf and centrifuged at 1500 rcf for 1 min. Supernatant was removed, cells were resuspended in 1 mL PBS and the mixture transferred to a new 1.5 mL eppendorf and incubated for 10 min. Cells were then centrifuged at 1500 rcf for 1 min, PBS aspirated, and the cells resuspended in 0.6 mL growth media. The suspensions were split into 3×200 μL aliquots and added to a 96-well plate. Cells were incubated for 10 min followed by centrifugation at 800 rcf for 5 min. Media was removed, cells were resuspended in 200 μL of media and transferred into a new 96 well plate and incubated for further 10 min. For the last washing step, cells were incubated for 20 min prior to centrifugation at 800 rcf for 1 min and aspiration of growth media. Cells were then resuspended in 200 μL of growth media incubated for 48 h (96 well plate). Cells were transferred to a new U-bottom 96-well plate pre-coated with αCD3 stimulant (2.5 μg mL⁻¹) and further incubated for 24 h. Supernatant and cells were centrifuged at 800 rcf for 5 min and 120 μL of supernatant was collected and analysed using MSD IFNγ kit as detailed above for the human whole-blood assay. All incubations were performed at 37° C. and 5% CO₂. The human biological samples were sourced ethically and their research use was in accord with the terms of the informed consents.

### Cellular Washout Data

The IC₅₀ curve 48 h after washing cells to remove compound followed, closely the curve without washing the cells. This confirms a duration of action of at least 48 h for Example 7 at PI3Kδ in CD4+T cells, supporting an irreversible covalent mode of action. Furthermore, this may provide a method of providing long-lasting treatment for PI3Kδ-related conditions. The experiment was repeated using five donors (for each donor: N=3 replicates for washout, and N=2 replicates for non-wash condition), and results are depicted as the mean±s.e.m.

What is claimed is:

1. A compound of formula (I) 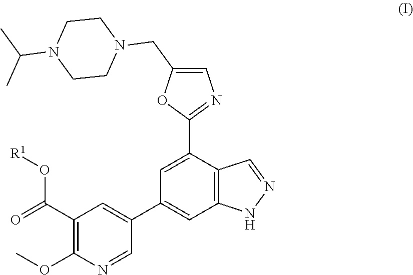
wherein
R¹ is hydrogen, C₁₋₆alkyl or phenyl, wherein the C₁₋₆alkyl is optionally substituted by —CF₃ and the phenyl is optionally substituted by one or two substituents independently selected from C₁₋₆alkyl, C₁₋₆alkoxy, halogen, —CN, —CF₃, —CO₂R², —CO₂NHR³, —NR⁴R⁵, —NO₂ and —SF₅,
R² to R⁵ are each independently selected from hydrogen and C₁₋₆alkyl,
or a salt thereof.

2. A compound according to claim 1, or a salt thereof, wherein R¹ is phenyl optionally substituted by one or two substituents independently selected from C₁₋₆alkyl, C₁₋₆alkoxy, halogen, —CN, —CF₃, —CO₂R², —CO₂NHR³, —NR⁴R⁵, —NO₂ and —SF₅.

3. A compound according to claim 1, or a salt thereof, wherein R¹ is phenyl optionally substituted by one or two substituents independently selected from C₁₋₆alkyl, C₁₋₆alkoxy, halogen, —CF₃ and —NO₂.

4. A compound which is:
methyl 5-(4-(5-((4-isopropylpiperazin-1-yl)methyl)oxazol-2-yl)-1H-indazol-6-yl)-2-methoxynicotinate,
5-(4-(5((4-isopropylpiperazin-1-yl)methyl)oxazol-2-yl)-1H-indazol-6-yl)-2-methoxynicotinic acid;
2-nitrophenyl 5-(4-(5-((4-isopropylpiperazin-1-yl)methyl)oxazol-2-yl)-1H-indazol-6-yl)-2-methoxynicotinate,
4-nitrophenyl 5-(4-(5-((4-isopropylpiperazin-1-yl)methyl)oxazol-2-yl)-1H-indazol-6-yl)-2-methoxynicotinate,
2-(trifluoromethyl)phenyl 5-(4-(5-((4-isopropylpiperazin-1-yl)methyl) oxazol-2-yl)-1H-indazol-6-yl)-2-methoxynicotinate,
4-(trifluoromethyl)phenyl 5-(4-(5-((4-isopropylpiperazin-1-yl)methyl)oxazol-2-yl)-1H-indazol-6-yl)-2-methoxynicotinate,
4-fluorophenyl 5-(4-(5-((4-isopropylpiperazin-1-yl)methyl)oxazol-2-yl)-1H-indazol-6-yl)-2-methoxynicotinate,
phenyl 5-(4-(5-((4-isopropylpiperazin-1-yl)methyl)oxazol-2-yl)-1H-indazol-6-yl)-2-methoxynicotinate,
2,4-dimethylphenyl 5-(4-(5-((4-isopropylpiperazin-1-yl)methyl)oxazol-2-yl)-1H-indazol-6-yl)-2-methoxynicotinate,
4-methoxyphenyl 5-(4-(5-((4-isopropylpiperazin-1-yl)methyl)oxazol-2-yl)-1H-indazol-6-yl)-2-methoxynicotinate,
2,2,2-trifluoroethyl 5-(4-(5-((4-isopropylpiperazin-1-yl)methyl)oxazol-2-yl)-1H-indazol-6-yl)-2-methoxynicotinate,
or a salt thereof.

5. A compound according to claim 1 in the form of a pharmaceutically acceptable salt thereof.

6. A pharmaceutical composition comprising a compound as defined in claim 1, or a pharmaceutically acceptable salt thereof, and one or more pharmaceutically acceptable excipients.

7-9. (canceled)

10. A method of treating a disorder mediated by inappropriate PI3-kinase activity comprising administering a safe and effective amount of a compound as defined in claim 1, or a pharmaceutically acceptable salt thereof, to a patient in need thereof.

11. A method according to claim 10 wherein the disorder mediated by inappropriate PI3-kinase activity is a respiratory disease, a ciliopathy, a bacterial infection or bacterial exacerbation of a respiratory condition or lung damage, a viral infection or viral exacerbation of a respiratory condition or lung damage, a non-viral respiratory infection, an allergic disease, an autoimmune disease, an inflammatory disorder, diabetes, a cardiovascular disease, a hematologic malignancy, a neurodegenerative disease, pancreatitis, multiorgan failure, kidney disease, platelet aggregation, cancer, sperm motility, transplantation rejection, graft rejection, lung injury, pain, fibrotic disease, depression or a psychotic disorder.

12. A method according to claim 10 wherein the disorder mediated by inappropriate PI3-kinase activity is a respiratory disease.

13. A method according to claim 10 wherein the disorder mediated by inappropriate PI3-kinase activity is asthma.

14. A method according to claim 10 wherein the disorder mediated by inappropriate PI3-kinase activity is COPD.
